(19) United States
(12) Patent Application Publication
(10) Pub. No.: US 2025/0186798 A1
(43) Pub. Date: Jun. 12, 2025

(54) HEAD SUPPORT GUIDE SYSTEM FOR TRANSCRANIAL MAGNETIC STIMULATION

(71) Applicant: Neuronetics, Inc., Malvern, PA (US)

(72) Inventors: Kenneth Ghiron, Malvern, PA (US); Michael Black, Phoenixville, PA (US)

(73) Assignee: Neuronetics, Inc., Malvern, PA (US)

(21) Appl. No.: 18/977,425

(22) Filed: Dec. 11, 2024

Related U.S. Application Data
(60) Provisional application No. 63/608,565, filed on Dec. 11, 2023.

(51) Int. Cl. A61N 2/02; A61N 2/00

(52) U.S. Cl. CPC A61N 2/02; A61N 2/006

(57) ABSTRACT

A method, system, and apparatus for a head guide support system for use in measuring a transcranial magnetic stimulation (TMS) treatment coil on a patient's head are described herein. The coil positioning apparatus, also called a coil positioning device or a coil angle guide, may include a support structure supported by the patient's head; a first instrument configured to indicate a position angle for placement of the TMS coil; a second instrument configured to indicate a distance from the support structure for placement of the TMS coil; and/or a third instrument configured to indicate an orientation angle for rotation of the TMS coil.

### CROSS-REFERENCE TO RELATED APPLICATIONS

This application claims the benefit of U.S. Provisional Application No. 63/608,565, filed Dec. 11, 2023, the disclosure of which is incorporated herein by reference in its entirety.

### BACKGROUND

A number of medical ailments may be treated and/or diagnosed through the application of a magnetic field to an afflicted portion of a patient's body. Neurons and muscle cells may be a form of biological circuitry that carry electrical signals and respond to electromagnetic stimuli. When a conductive wire loop is passed through a magnetic field or is in the presence of a changing magnetic field, an electric current may be induced in the wire. The same principle may hold true for conductive biological tissue. When a changing magnetic field is applied to a portion of the body, neurons may be depolarized and stimulated. Muscles associated with the stimulated neurons may contract as though the neurons were firing by normal causes.

A nerve cell or neuron may be stimulated in a number of ways, for example, transcutaneously via transcranial magnetic stimulation (TMS). TMS may use a rapidly changing magnetic field to induce a current on a nerve cell, without having to cut or penetrate the skin. The nerve may “fire” when a membrane potential within the nerve rises with respect to its normal negative ambient level of approximately −90 mV, for example, depending on the type of nerve, local pH of the surrounding tissue, and/or peripheral nerve stimulation.

Typical TMS treatment apparatuses generate pulsed magnetic fields that induce currents in electrically sensitive cells (e.g., nerve cells or neurons). These induced currents typically form a complete circuit in the body, such that a path of zero current through the body is created. The currents induced by a TMS treatment apparatus typically drop off to zero in approximately the middle of this path. The rate of this current drop off may be slowed, for example by spreading the current density generated the TMS apparatus over a wide surface area. However, employing this approach may concentrate return currents, which may lead to higher rates of undesirable side effects (e.g., the stimulation of untargeted regions of a subject's brain).

A typical TMS treatment apparatus may include one or more electrically conductive stimulating coils. Such coils may be configured (e.g., wound) in a single layer, such that the coils may be disposed as close as possible to the tissue that is to be stimulated. Such coils may be capable of stimulating brain tissue at a desirable depth relative to the skull. However, such coils are traditionally only configured to stimulation a single location within a subject, and in order to stimulation multiple locations, multiple coils, which may be large and cumbersome, may be required.

Prior to treatment, the user's treatment location may first be determined. For TMS, for example, the treatment location is typically determined based on the subject's motor threshold (MT) position, which itself is determined by moving the coil near a predicted area determined by patient anatomical landmarks until the desired motor response (e.g., thumb twitch) is achieved. The MT position (e.g., MT location) is marked, for example, with an ink mark on the subject's head, skull cap, and/or other covering worn by the patient. In the case of using the TMS coil for treatment of depression, for example, the TMS therapy position is determined by moving the coil from the MT position along a line in the anterior direction a prescribed distance (e.g., a distance of approximately 4.0 cm to 6.0 cm) to identify the treatment location on the subject. Notably, the MT position and treatment location are not necessarily the same point.

Under the present methods, determination of the MT position and corresponding treatment location must occur prior to each treatment. Accordingly, the current treatment methods require a system that can easily and accurately capture the measurements as well as quickly reproduce the measurements without repeated tedious measuring. This eliminates the need to remeasure with each subsequent treatment.

### SUMMARY

A method, system, and apparatus for a head guide support system for use in measuring a transcranial magnetic stimulation (TMS) treatment coil on a patient's head are described herein.

The coil positioning apparatus, also called a coil positioning device, may include a support structure supported by the patient's head; a first instrument configured to indicate a position angle for placement of the TMS coil; a second instrument configured to indicate a distance from the support structure for placement of the TMS coil; and/or a third instrument configured to indicate an orientation angle for rotation of the TMS coil.

The coil positioning device may identify a location of the TMS coil for TMS treatment based on the distance and/or the position angle relative to a reference point defined by the support structure. The coil positioning device may identify an orientation of the TMS coil for TMS treatment based on the orientation angle.

The support structure may include a first elongated arm configured to be supported by a first ear of the patient; a second elongated arm configured to be supported by a second ear of the patient; and a crossbar residing between the first elongated arm and the second elongated arm. The support structure, the crossbar, and/or the first instrument may define a reference point for measuring the position angle, the distance, and the orientation angle. The crossbar may be connected to the first elongated arm by a first hinge and/or the second elongated arm by a second hinge.

The first instrument may be coupled to the crossbar and/or at the reference point. The third instrument may be coupled to a distal end of the second instrument. The third instrument may include a reference point for measuring the orientation angle. The second instrument may be a tape measure. The measured distance may be an arc length.

Also disclosed herein is another example of a support structure supported by the patient's head. The support structure may include a first elongated arm; a second elongated arm; and a crossbar residing between the first elongated arm and/or the second elongated arm. The crossbar may include a first instrument for measuring a position angle for placement of the TMS coil to conduct a TMS treatment session. The crossbar may define a reference point for measuring the position angle.

A second instrument may be coupled to the support structure. The second instrument may be for measuring a distance from the support structure to the TMS coil to conduct the TMS treatment session. The second instrument may include a third instrument at a distal end of the second instrument for measuring an orientation angle of the TMS coil to conduct the TMS treatment session.

The position angle may represent an angular offset between a line located within a median plane of the patient that intersects with the reference point and a line that intersects the reference point and a motor threshold location of the patient.

The orientation angle may represent an angular offset between a line that intersects the motor threshold location of the patient and the TMS and a line that intersects the reference point and the motor threshold location of the patient.

The third instrument may define a flat edge. The orientation angle may represent an angular offset between a line that intersects the flat side of the third instrument and the motor threshold location of the patient and a line that intersects the motor threshold location of the patient and the TMS coil (e.g., an edge of the TMS coil) and a line that intersects the reference point and the motor threshold location of the patient. The flat edge may rest next to an edge of the TMS coil.

The support structure may align the reference point with the patient's nasion. The first instrument may include a protractor. The first instrument may be coupled to the crossbar at the reference point. The second instrument may indicate the position angle as measured by the first instrument. The support structure may be made of plastic.

The first instrument may include indicia that represent a plurality of different position angles. The second instrument may include a center line that traverses a length of the second instrument and/or may indicate the position angle for placement of the TMS coil. The first instrument and/or the second instrument may be comprised of a transparent material.

The second instrument may include a first end and/or a second end. The second end may be coupled to the third instrument for measuring the orientation angle. The crossbar may include a connector that may receive the second instrument. The crossbar may allow for the second instrument to be slidably adjusted to indicate the distance from the support structure to the TMS coil. The connector may include indicia to identify the distance from the support structure to the TMS coil. The connector may include an aperture, and the first end of the second instrument may pass through the aperture.

The second instrument may include a second reference point for measuring the orientation angle. The second instrument may indicate the orientation angle via the third instrument. The third instrument may include indicia that represent a plurality of different orientation angles. The second instrument may include a center line that traverses a length of the second instrument and/or may indicate the orientation angle.

The third instrument may include a first flat surface positioned along a flat sidewall of the coil, and/or a second flat surface that extends approximately perpendicularly from the first flat surface. The second flat surface may include indicia used to determine the orientation angle. A first ear of the patient may support the first elongated arm and/or a second ear of the patient may support the second elongated arm. The template may indicate a motor threshold point relative to the edge of the TMS coil. The template may determine a treatment location point for the TMS based on the motor threshold location, the reference point, and/or a predetermined distance. The predetermined distance may be equal to or between 4.0 cm and 6.0 cm.

The device may include head covering (e.g., a skull cap) placed on the patient's head. The edge of the TMS coil may be marked on the skull cap for determining the motor threshold location for TMS.

Also disclosed herein are a device and a method for performing a TMS treatment session. The device and method may position a treatment coil proximate to a motor threshold location on a head of a patient. The device and method may generate a mark on a head covering at a front edge of the treatment coil. The device and method may record the motor threshold location on the head covering based on the mark at the front edge. The device and method may determine a motor threshold to reference line based on the recorded motor threshold location and a support structure, a position angle measurement instrument, and/or a location on a face of the patient. The device and/or method may determine a treatment location for the patient along the motor threshold to reference line. The device and method may determine an orientation angle for the treatment coil using an orientation angle measurement instrument. The device and method may place the treatment coil proximate to the treatment location. The device and method may determine a linear distance measurement for the treatment coil and/or a position angle of the treatment coil. The device and method may perform a TMS procedure using the treatment coil and/or based on the orientation angle, the linear distance measurement, and/or the position angle.

The location may be a treatment location and/or a motor threshold location on the head of the patient. The motor threshold to reference line is based on the recorded motor threshold location and/or the support structure. The motor threshold to reference line may be based on the recorded motor threshold location and the position angle measurement instrument. The motor threshold to reference line may be based on the recorded motor threshold location and/or the location on the face of the patient. The location on the face of the patient may comprise a nasion, a philtrum, a nose, and/or a space between eyes of the patient. The treatment location may be determined using a measurement template.

The device and method may move the measurement template from the recorded motor threshold location along the MT-to-reference line a predetermined distance. The predetermined distance may be 4.0 cm to 6.0 cm or 5.0 cm or 5.5 cm. A center point of the measurement template may overlaps the treatment location after the measurement template is moved the predetermined distance along the MT-to-reference line from the recorded motor threshold location.

The orientation angle may be an angle of the treatment coil relative to a reference point. The reference point may be located on a support structure, a position angle measurement instrument, and/or a face of the patient. The orientation angle may be determined by placing the orientation angle measurement instrument against a measurement template such that the orientation angle measurement instrument contacts the measurement template. The device and method may comprise recording the treatment location on the head covering.

The linear distance measurement may be determined between the treatment coil and a support structure. The device and method may comprise placing a support structure on the patient's head, wherein the orientation angle, the linear distance measurement. The position angle for the treatment coil may be determined using the support structure. The device and method may perform subsequent TMS treatments on the patient using the support structure and/or based on the orientation angle, the linear distance measurement, and the position angle. The subsequent TMS treatments may be performed without needing to recalculate the orientation angle, the linear distance measurement, and/or the position angle. The subsequent TMS treatments may be performed without needing to record the motor threshold location on the head covering. The device and method may determine the motor threshold to reference line, and/or determine the treatment location along the motor threshold to reference line.

Also disclosed herein are a device and a method for performing a transcranial magnetic stimulation (TMS) treatment session. The device and a method may determine an orientation angle for a treatment coil using an orientation angle measurement instrument of a support structure. The device and a method may determine a linear distance measurement for the treatment coil using the support structure. The device and a method may determine a position angle of the treatment coil using the support structure. The device and a method may perform a TMS procedure using the treatment coil and based on the orientation angle, the linear distance measurement, and/or the position angle.

The support structure may be supported by a head of the patient. The support structure may comprise: a first elongated arm configured to be supported by a first ear of the patient; a second elongated arm configured to be supported by a second ear of the patient; and/or a crossbar residing between the first elongated arm and the second elongated arm. The support structure may comprise: a first instrument configured to indicate a position angle for placement of the TMS coil; a second instrument configured to indicate a distance from the support structure for placement of the TMS coil; and/or a third instrument configured to indicate an orientation angle for rotation of the TMS coil.

### BRIEF DESCRIPTION OF THE DRAWINGS

FIG. 1 is a diagram of an example of a treatment or diagnostic system.

FIG. 2 is a block diagram illustrating an example of a magnetic stimulation system.

FIG. 3A is a diagram of an example head support guide system for a transcranial magnetic stimulation (TMS) coil configured to be worn on a patient's head.

FIG. 3B depicts the example head support guide in contact with the treatment coil and being worn by a patient.

FIG. 3C depicts a front perspective view of the example head support guide with an alternate orientation angle measurement instrument when not worn by the patient.

FIG. 3D depicts a top-down plan view of the example head support guide when not worn by the patient.

FIG. 3E depicts a right-side view of the example head support guide with the alternate orientation angle measurement instrument.

FIG. 3F provides a detailed view, from the bottom/front perspective, of the position angle measurement instrument, linear measurement instrument, and alternative orientation angle measurement instrument coupled together of the example head support guide.

FIG. 3G depicts a right-side view of the alternative orientation angle measurement instrument of the example head support guide.

FIG. 3H depicts a front perspective view of another alternative orientation angle instrument.

FIG. 3I depicts a front/top perspective view of another alternative orientation angle instrument before the ears contact the surface of a patient's head.

FIG. 3J depicts a front/top perspective view of another alternative orientation angle instrument after the ears contact the surface of a patient's head.

FIG. 4A depicts an overhead perspective of the head support guide system and TMS coil worn on the patient's head.

FIG. 4B depicts an overhead perspective of a template used to establish a motor threshold (MT) position.

FIG. 4C depicts an overhead perspective of a template used to measure establish a treatment location.

FIG. 4D depicts an overhead perspective of a template used to establish lines for use in measuring a TMS coil orientation angle.

FIG. 4E depicts a head support guide system fitted on a patient's head for use in measuring a TMS coil position angle.

FIG. 5 is a flowchart depicting an example operation of the head support guide and method of obtaining critical measurements that obviate the need for repeated use of the head support guide.

FIG. 6 depicts an alternative head support guide system wherein the patient uses removable eyeglasses.

FIG. 7 depicts an alternative device for establishing measurements for reproducing placement of the treatment coil.

FIG. 8 depicts an alternative method for establishing measurements for reproducing placement of the treatment coil.

### DETAILED DESCRIPTION

The following discussion omits or only briefly describes conventional features of treatment or diagnostic systems, which are apparent to those skilled in the art. It is noted that various embodiments are described in detail with reference to the drawings, in which like reference numerals represent like parts and assemblies throughout the several views. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an,” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

In 1831, Michael Faraday discovered that the magnitude of an electric field induced on a conductor is proportional to the rate of change of magnetic flux that cuts across the conductor. Faraday's law, well known to those skilled in the art, may be represented as E˜−(A*dB/dt), where E is the induced electric field in volts/meter and dB/dt is the time rate of change of magnetic flux density in Tesla/second. In other words, the amount of electric field induced in an object, such as a conductor, may be determined using two factors: the area density and the time rate of change of the flux. The greater the flux density and its derivative, the greater the induced electric field and resulting current density. Magnetic flux may be a function of distance. For example, because the magnetic flux density may decrease in strength with relation to the distance from the source of the magnetic field (e.g., 1/r³, 1/r⁵, or the like), the flux density may be greater the closer the conductor is to the source of the magnetic field. When the conductor is a coil, the current induced in the coil by the electric field may be Increased in proportion to the number of turns of the coil.

An overview of an example operation and application of a magnetic system in which aspects of the various embodiments may be implemented may be provided. The magnitude of an electric field induced on a conductor may be proportional to the rate of change of magnetic flux density across the conductor. When an electric field is induced in a conductor, the electric field may create a corresponding current flow in the conductor. The current flow may be in the same direction of the electric field vector at a given point. The peak electric field may occur when the time rate of change of the magnetic flux density is the greatest and may diminish at other times. During a magnetic pulse, the current may flow in a direction that tends to preserve the magnetic field (e.g., Lenz's Law).

Certain parts of the anatomy (e.g., nerves, tissue, muscle, brain) may act as a conductor and may carry electric current when a pulsed magnetic field is applied. The pulsed magnetic field may be applied to these parts of the anatomy transcutaneously. For example, in the context of transcranial magnetic stimulation (TMS), a time-varying magnetic field may be applied across the skull to create an electric field in the brain tissue, which may produce a current. If the induced current is of sufficient density and/or duration, neuron action potential may be reduced to the extent that the membrane sodium channels open and an action potential response is created. An impulse of current may be propagated along the axon membrane that transmits information to other neurons via modulation of neurotransmitters. Such magnetic stimulation may acutely affect glucose metabolism and local blood flow in cortical tissue. In the case of major depressive disorder, neurotransmitter dysregulation and abnormal glucose metabolism in the prefrontal cortex and the connected limbic structures may be a likely pathophysiology. Repeated application of magnetic stimulation to the prefrontal cortex may produce chronic changes in neurotransmitter concentrations, metabolism, and/or nerve changes to stimulation thresholds, for example, such that depression may be alleviated.

Non-cortical neurons (e.g., cranial nerves, peripheral nerves, sensory nerves) may be stimulated by an induced electric field. For example, peripheral nerves may be intentionally stimulated to diagnose neuropathologies, for example, by observing response times and conduction velocities in response to a pulsed magnetic field induced stimulus. Discomfort and/or pain may result if the induced electric field applied to a peripheral and/or cranial nerve is very intense, and/or focused on a small area of the nerve. This discomfort may be diminished, for example, by intentionally over-stimulating the sensory nerves in the affected nerve bundle so that they can no longer respond to external pain stimuli, or by reducing the intensity and/or focus of the induced electric field that is causing the pain sensation.

Transcranial magnetic stimulation may not be limited to treatment of depression. Transcranial magnetic stimulation may be used to treat a patient, such as a human, for example, suffering from epilepsy, schizophrenia, Parkinson's disease, Tourette's syndrome, amyotrophic lateral sclerosis (ALS), multiple sclerosis (MS), Alzheimer's disease, attention deficit/hyperactivity disorder, obesity, bipolar disorder, anxiety disorders (e.g., panic disorder with and without agoraphobia, social phobia also known as social anxiety disorder, acute stress disorder and/or generalized anxiety disorder), post-traumatic stress disorder (one of the anxiety disorders in DSM), obsessive compulsive disorder (e.g., one of the anxiety disorders in DSM), pain (such as, for example, migraine and trigeminal neuralgia, as well as chronic pain disorders, including neuropathic pain, e.g., pain due to diabetic neuropathy, post-herpetic neuralgia, and idiopathic pain disorders, e.g., fibromyalgia, regional myofascial pain syndromes), rehabilitation following stroke (neuro plasticity induction), tinnitus, stimulation of implanted neurons to facilitate integration, substance-related disorders (e.g., dependence, abuse and withdrawal diagnoses for alcohol, cocaine, amphetamine, caffeine, nicotine, cannabis and the like), spinal cord injury and regeneration/rehabilitation, stroke, head injury, traumatic brain injury, sleep deprivation reversal, primary sleep disorders (primary insomnia, primary hypersomnia, circadian rhythm sleep disorder), cognitive enhancements, short/long term memory, mild cognitive impairment, dementias, premenstrual dysphoric disorder (PMS), drug delivery systems (changing the cell membrane permeability to a drug), induction of protein synthesis (induction of transcription and translation), stuttering, aphasia, dysphagia, essential tremor, autism spectrum disorders, and/or eating disorders (such as bulimia, anorexia, and binge eating).

A device may take advantage of the above principles to induce an electric field used in a variety of applications. For example, a magnetic device may be used for electrical stimulation of the anatomy. While the discussion herein focuses on magnetic devices used in connection with magnetic stimulation of anatomical tissue, as well as the head guide support system that simplifies the repeated uses of these magnetic devices, a magnetic device may be utilized in any field of endeavor. Further, as the devices provided herein are described with reference to magnetic stimulation such as, for example, TMS, the devices may be used for any treatment or diagnostic procedure including, a motor threshold detection procedure, or MT procedure, as described herein.

A ferromagnetic core may be used in connection with a magnetic device to produce a magnetic field. For example, a ferromagnetic core may include an arc-shaped (e.g., approximately hemispherical) magnetic material. A ferromagnetic core may include a highly saturable magnetic material having a magnetic saturation of at least 0.5 Tesla. A ferromagnetic core may be shaped to optimize the magnetic field distribution in the treatment area. For example, such a magnetic field may be for purposes of carrying out transcranial magnetic stimulation such as, for example, TMS, Repetitive TMS (rTMS), Magnetic Seizure Therapy (MST), deep TMS (dTMS), controlled and/or varied pulse shape TMS (cTMS), reduction of peripheral nerve discomfort, etc. Although examples described herein may be discussed in connection with TMS and rTMS, the examples described herein may be utilized in connection with any type of magnetic stimulation, such as transcranial magnetic stimulation, for example. Furthermore, the embodiments presented herein are not limited to the use of ferromagnetic core magnetic stimulation systems, as other core materials may be used such as, for example, an air core.

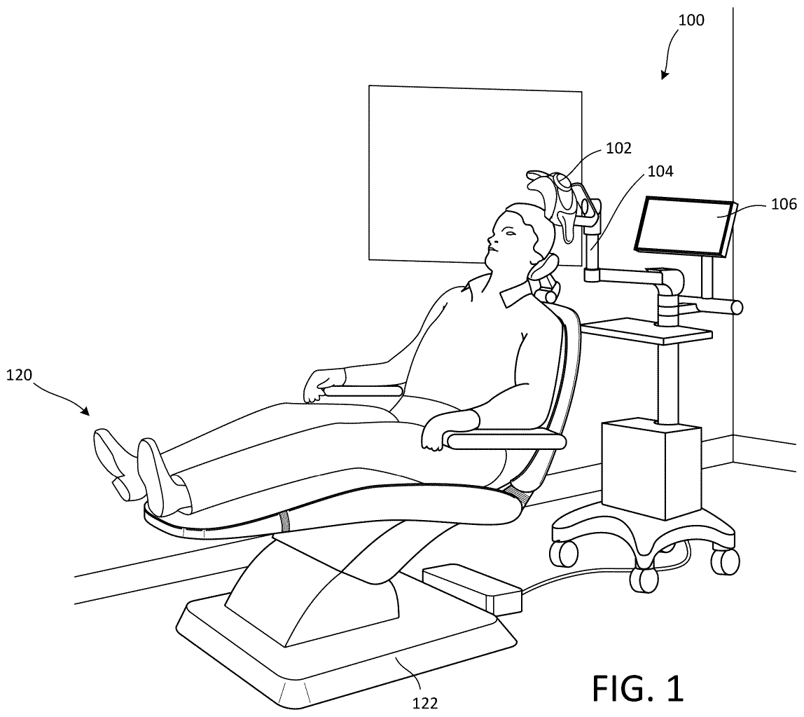

FIG. 1 is a diagram of an example of a treatment or diagnostic system 100. The treatment or diagnostic system 100 may comprise a processor (not shown), a power supply (not shown), memory (not shown), a transceiver, (not shown), a treatment coil 102 (e.g., stimulation coil), an articulating arm 104, a display device 106, a human subject positioning apparatus 122, and/or a motion detection device, such as a motor threshold detection device.

The treatment system 100 may be stationary or movable. For example, the treatment system 100 may be integrated into a movable cart, for example, as shown in FIG. 1. In one or more examples, the treatment system 100 may be a TMS treatment system (e.g., NeuroStar®) and/or any other therapeutic and/or diagnostic procedure system.

The treatment coil 102 (e.g., an electromagnet) may be used to administer a therapeutic and/or diagnostic procedure to a human subject 120, for example, TMS. Example treatment coils 102 may include one or more treatment coils and one or more ferromagnetic components that are configured to be disposed proximate to corresponding ones of the one or more treatment coils. The one or more treatment coils and ferromagnetic components of each TMS device may cooperatively generate a magnetic field that exhibits one or more characteristics that differ from those of a magnetic field that is generated by the one or more treatment coils alone. For example, the treatment system 100 may include a drive circuit (not shown) that may be configured to cause the treatment coil 102 to generate a magnetic field. Examples of a drive circuit that may be used in the treatment system 100 are described in U.S. Pat. No. 7,744,523, which is hereby incorporated by reference in its entirety. The processor of the treatment system 100 may be configured to generate one or more drive signals (e.g., via the drive circuit) that are configured to cause the treatment coil 102 to generate a pulsating magnetic field. The pulsating magnetic field may be defined by one or more pulses in one or more pulse bursts. The processor of the treatment system 100 may be configured to send signals to a motor threshold device (e.g., to a user interface associated with the motor threshold device) that indicate the timing and/or the power of the pulses of the pulsating magnetic field (e.g., that indicate the timing and power of the drive signals used to generate the pulsating magnetic field).

Although illustrated to include the treatment coil 102 and described primarily with respect to TMS, the treatment system 100 may include any device for administration of therapeutic and/or diagnostic procedure of the human subject. In some examples, the treatment system 100 may be used for a diagnostic procedure (e.g., solely for a diagnostic procedure). Examples of TMS coils are described in are described in U.S. Pat. Nos. 7,824,324 and 11,000,693, the contents of which are incorporated herein by reference in their entirety.

The processor (e.g., controller) of the treatment system 100 may be a general purpose processor, a special purpose processor, a conventional processor, a digital signal processor (DSP), a plurality of microprocessors, one or more microprocessors in association with a DSP core, a microcontroller, Application Specific Integrated Circuits (ASICs), Field Programmable Gate Array (FPGAs) circuits, any other type of integrated circuit (IC), a state machine, and the like. The processor may perform signal coding, data processing, power control, input/output processing, and/or any other functionality that enables the treatment system 100 to operate. The processor may be integrated together with one or more other components of the treatment system 100 in an electronic package or chip.

The processor of the treatment system 100 may be coupled to and may receive user input data from and/or output user input data to the treatment coil 102, the articulating arm 104, the display device 106 (e.g., a liquid crystal display (LCD) display unit or organic light-emitting diode (OLED) display unit), and/or the human subject positioning apparatus 122. The processor may access information from, and store data in, any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The processor may access information from, and store data in, memory that is not physically located within the treatment system 100, such as on a server (not shown).

The memory may comprise a computer-readable storage media or machine-readable storage media that maintains computer-executable instructions for performing one or more as described herein. For example, the memory may comprise computer-executable instructions or machine-readable instructions that include one or more portions of the procedures described herein. The processor of the treatment system 100 may access the instructions from memory for being executed to cause the processor to operate as described herein. The memory may comprise computer-executable instructions for executing configuration software. For example, the computer-executable instructions may be executed to perform, in part and/or in their entirety, one or more procedures as described herein. Further, the memory may have stored thereon one or more settings and/or control parameters associated with the treatment system 100, a motor threshold detection device, and/or the user interface for a motor threshold detection device.

The processor may receive power from the power supply, and may be configured to distribute and/or control the power to the other components in the treatment system 100. The power supply may be any suitable device for powering the treatment system 100.

The human subject 120 may be positioned within the human subject positioning apparatus 122. The human subject positioning apparatus 122 may be a chair, recliner, bed, stool, and/or the like. When performing treatment, the treatment coil 102 may be situated such that the human subject's head is positioned under the treatment coil 102. The treatment coil 102 may be adjusted by means of the articulating arm 104 and/or the like. The human subject positioning apparatus 122 may include one or more arms, such as a right arm and a left arm as shown in FIG. 1. Although not illustrated in FIG. 1 and as described in more detail below, the right and/or left arm of the human subject positioning apparatus 122 may include a motor threshold detection device (not pictured in FIG. 1). The motor threshold detection device may be permanently or removable mounted to the right arm and/or the left arm of the human subject positioning apparatus 122. The motor threshold detection device may be connected to the processor of the treatment system 100, for example, via an electrical connection and/or a wireless connection. In some examples, the motor threshold detection device may include a dedicated display device (not shown), while in other examples, the motor threshold detection device may use the display device 106 of the treatment system 100.

The motor threshold detection device may include one or more movement detection sensors, such as accelerometers, to detect movement of one or more body parts of the human subject 120, such as, but not limited to, fingers, a foot, or the like. The treatment system 100 (e.g., the motor threshold detection device) may be configured to detect movement of the human subject's 120 fingers that is a result from a magnetic pulse generated by the treatment coil 102. For instance, the treatment system 100 may use the motor threshold detection device to detect movement of the fingers of the human subject 120 within a certain time period after a pulse is generated (e.g., movement that occurs within 0.25 seconds after the generation of the magnetic pulse). The treatment system 100 may ignore movements outside of the time period. It is noted that the examples provided herein correspond to the motor threshold detection device of the treatment system 100 detecting movement of one or more fingers of the human subject 120. However, it should be understood that the motor threshold detection device may detect other bodily movements of the human subject 120 (e.g., a foot of the human subject 120), and examples of detecting bodily movement of the human subject 120 are not limited to only detecting movement of one or more fingers of the human subject 120 (e.g., when treating other disorders, such as OCD and/or PTSD).

The treatment system 100 may comprise one or more computer software applications stored in memory of the treatment system and/or running on the processor. The computer software applications may provide a system graphical user interface (GUI) (e.g., a TMS system GUI) on the display device 106. The computer software applications may incorporate work flow management to guide a technician through the therapeutic and/or diagnostic procedure, and/or supervise and/or control one or more subsystems of the treatment system 100. For example, the computer software applications may control internal system functions, monitor the system status to ensure safe operation, and/or provide the user with a graphical means to manage the preparation for and/or the administration of the therapeutic and/or diagnostic procedure.

Interaction with the computer software applications may be provided via a user interface. In one or more embodiments, the user interface device may be the display device 106, which may be a touch screen display. The display device 106 may include touch activated images of alphanumeric keys and/or buttons for user interaction with the treatment system 100. The display device 106 may provide graphic representations of the system activity, messages, and/or alarms. Interactive buttons, fields, and/or images may be displayed via the display device 106, and may enable the technician to direct and/or interact with system functions, for example, such as entering data, starting and stopping the procedure, running diagnostics, adjusting positioning and/or configuration of the treatment coil 102, adjusting the position of one or more sensor(s), and/or the like.

The treatment system 100 may be used for any therapeutic and/or diagnostic procedure. For example, the treatment system may be used for TMS, transcranial direct current stimulation (tDCS), electroencephalography (EEG), deep brain stimulation (DBS), a diagnostic procedure, and/or the like. For example, the treatment system 100 may be used for any therapeutic and/or diagnostic procedure that includes the placement of electrodes, sensors, probes, and/or the like on a human subject, such as on the surface of a human subject's head. The treatment system 100 may have further application in neurosurgery, wherein the treatment system may map regions of interest I the patient's brain. These maps may help physicians when operating on the brain (e.g., removal of tumors, performing a brain implant, etc.) Although described with reference to a head model, the treatment system 100 may be configured to generate a model of any part of the human subject 120, such as, but not limited to, the arm, neck, chest, leg, foot, and/or the like. Example methods of using the treatment system 100 to determine the human subject's 120 MT position and/or treatment location are described in U.S. Pat. Nos. 7,104,947 and 9,884,200, the contents of which are incorporated herein by reference in their entirety.

Further, as noted above, prior to TMS treatment, the treatment location of the human subject 120 may first be determined. For TMS, for example, the treatment location is typically determined based on the subject's MT position, which itself is determined by moving the coil near a predicted area determined by patient anatomical landmarks until the desired motor response (e.g., thumb twitch) is achieved. This process may be referred to as a motor threshold detection procedure, and may be part of the TMS treatment procedure (e.g., may be a preliminary step that is performed prior to generating one or more treatment pulse burst sets). In addition to detecting the location of the MT position, the treatment system 100 may also be used to detect the power of the pulses needed to cause the neurons at the MT position to be depolarized and stimulated (e.g., until the desired motor response in the human subject 120 (e.g., thumb twitch) is achieved). The treatment system 100 may determine the human subject's 120 MT position and power level using the motor threshold detection device (not pictured in FIG. 1).

Alternative means of obtaining the treatment location may include, but are not limited to, use of electroencephalogram (EEG) coordinates, the beam F3 method, use of magnetic resonance imaging (MRI) testing, and/or from Montreal Neurological Institute (MNI) coordinates.

After the location of the MT position and/or the power level of a test pulse are detected, the MT position and/or power level may be stored. For example, the MT position may be marked, for example, with an ink mark on the subject's head, or on a skull cap or other head covering worn on the head but under the coil. The MT position may be stored within the processor of the treatment system. Further, the power level of the test pulse may be stored, for example, in the memory of the treatment system 100. The treatment location may then be determined using the MT position. Consistent and accurate determination of this MT position leads to consistent and accurate determination of the treatment location. The MT position and treatment location are not necessarily the same point. Identification of the proper treatment location, whether using the MT position or not, ensures proper TMS treatment. Further, although the description is primarily described in context of determining the treatment location based on the MT position, in other examples the systems and methods described herein may determine the treatment location without the prior determination of the MT position and/or determine the treatment location by using one or more other coordinates or landmarks on the user's body (e.g., based on an anatomical landmark, image data (e.g., MRI data), 10/20 grid locations, etc.).

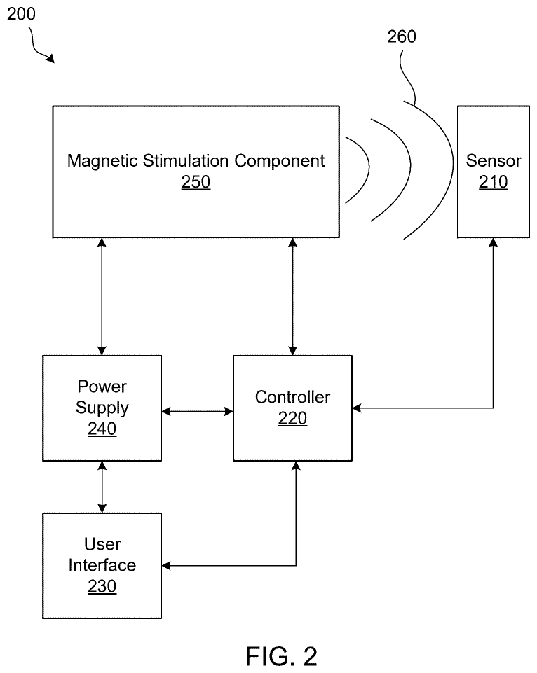

FIG. 2 is a block diagram illustrating an example of a magnetic stimulation system 200. The magnetic stimulation system 200 may be an example of the treatment system 100. The magnetic stimulation system 200 may comprise a sensor 210, a controller 220, a user interface 230, a power supply 240, and a magnetic stimulation component 250. The magnetic stimulation component 250 may be an example of the treatment coil 102 of the treatment system 100 of FIG. 1.

The magnetic stimulation component 250 may be configured to generate a pulsing magnetic field 260 to conduct magnetic stimulation therapy on a treatment area of a patient. The magnetic stimulation therapy may be, for example, TMS. TMS may refer to TMS, repetitive transcranial magnetic stimulation (rTMS), deep TMS (dTMS), cTMS, iTBS, cTBS, or the like. The magnetic stimulation component 250 may be a treatment coil. The magnetic stimulation component 250 may include a single treatment coil, multiple treatment coils, and/or an array of treatment coils. The treatment area may be the prefrontal cortex, for example. The magnetic stimulation component 250 may or may not include a core, such as a magnetic core (e.g., ferromagnetic core), for example. The pulsing magnetic field 260 may include one or more pulse bursts. A pulse burst (e.g., each pulse burst) of the pulsing magnetic field 260 may include one or more pulses.

The sensor 210 may be configured to generate a signal associated with a pulsing magnetic field 260. The sensor 210 may be placed between the magnetic stimulation component 250 and a treatment area of a patient. The sensor 210 may be configured to generate a signal associated with the pulsing magnetic field 260 of the magnetic stimulation component 250 (e.g., a signal induced by the pulsing magnetic field 260). For example, the sensor 210 may convert a physical property (e.g., the strength of pulsing magnetic field 260) into a corresponding electrical signal (e.g., a current signal or a voltage signal). As such, the sensor 210 may detect and/or measure a physical parameter of the pulsing magnetic field and generate a signal associated with the pulsing magnetic field using the detected/measured physical parameter. The generated signal may be a voltage signal, a current signal, and/or the like that may be proportional to a change in the pulsing magnetic field 260. For example, a current may be generated in the sensor 210 that may be proportional to the pulsing magnetic field 260. The sensor 210 may generate a voltage that may be proportional to the magnetic flux density (dB/dt) of the pulsing magnetic field 260.

The sensor 210 may include one or more of a conductive coil, a loop (e.g., having a number of turns based on the pulsing magnetic field), a Hall sensor, a magnetoresistive material, a Faraday effect sensor, a Kerr effect sensor, a flux gate sensor, an inductance change element, a nerve tissue response measurement device, an electric field sensor (e.g., in a conductive field), and/or the like. The sensor 210 may be configured to generate more than one signal, for example, more than one signal that is associated with the pulsing magnetic field 260 generated by the magnetic stimulation component 250.

The controller 220 may be any type of hardware, software, or combination thereof. The controller 220 may be configured to control one or more of the components of the magnetic stimulation system 200, such as the sensor 210, the user interface 230, the power supply 240, and/or the magnetic stimulation component 250, for example to conduct magnetic stimulation therapy. For example, the controller 220 may include a general purpose processor, a special purpose processor, a conventional processor, a digital signal processor (DSP), a plurality of microprocessors, one or more microprocessors in association with a DSP core, a microcontroller, any other type of integrated circuit (IC), a state machine, and/or the like.

The controller 220 may be configured to receive inputs from the user interface 230 and/or the sensor 210 to conduct magnetic stimulation therapy accordingly. For example, the controller 220 may perform signal coding, data processing, power control, input/output processing, and/or any other functionality that enables the controller 220 to operate the magnetic stimulation component for magnetic stimulation. The controller 220 may include a drive circuit that generates a drive signal for driving (e.g., powering, such as pulsing) the magnetic simulation component 250. In some examples, the drive circuit may be separate from the controller 220 and electrically coupled to the magnetic stimulation component 250.

Further, the controller 220 may be configured to alter the drive signal provided to the magnetic stimulation component 250 based on inputs received from the user interface 230 and/or the sensor 210. The controller 220 may be configured to estimate (e.g., measure) characteristics associated with the signal generated by the sensor 210 (e.g., associated with one or more peaks of the signal). The controller 220 may estimate a subset of the pulses of the signal or may estimate the signal continuously. By estimating characteristics of the signal, the controller 220 may estimate a model of what is occurring in the brain of a patient in response to the pulsing magnetic field.

Further, the controller 220 may determine whether a failure has occurred based on one or more characteristics of the signal generated by the sensor 210. In the event a failure is determined to have occurred, the controller 220 may enter a failure mode. In the failure mode, the controller 220 may pause the magnetic stimulation procedure, shut down the magnetic stimulation component 250, alert a user of the magnetic stimulation system 200, and/or alter a current applied to the magnetic stimulation component 250. For example, when the controller 220 enters the failure mode, the controller 220 may adjust the frequency at which it estimates characteristics of the generated signal. For example, the controller 220 may check for failures more frequently after a first failure is detected. Further, the magnetic stimulation system 200 may include an indicator that may indicate to a user of the magnetic stimulation system 200 that a failure has occurred. For example, the indicator may be a light, a speaker, an icon displayed on the user interface 230, and/or the like.

The user interface 230 may be any type of interface in which a user of the magnetic stimulation system 200 may initiate, adjust, and/or end the magnetic stimulation procedure. For example, the user interface may include a personal computer (PC), a keyboard, a mouse, a touchscreen, a wireless device, and/or the like, that allows for an interface between the user and the magnetic stimulation system 200.

The power supply 240 may be any type of power source that provides sufficient energy for the magnetic stimulation component 250 to generate the pulsing magnetic field 260 for its intended purpose, for example, for TMS, rTMS, MST or any other type of application. For example, the power supply 240 may be a conventional 120 or 240 VAC main power source.

The treatment coils described herein may be used as a treatment coil in a treatment or diagnostic system (e.g., the treatment coil 102 in the treatment or diagnostic system 100) and/or as a part of a magnetic stimulation component of a magnetic stimulation system (e.g., the magnetic stimulation component 250 of the magnetic stimulation system 200). Further, although described as driven by a single drive circuit, the treatment coils described herein may be driven by more than a single drive circuit, for example, driven by a drive circuit for each conductive element or a subset of the conductive elements of the treatment coil. Also, the conductive elements described herein may comprise any suitable material, such as Litz wire.

The treatment coils may include one or more ferromagnetic components. In some examples, the ferromagnetic component(s) may be referred to as a magnetic core. The ferromagnetic components may be comprised of any combination of a powdered magnetic material, a laminated magnetic material, an amorphous magnetic material, one or more of an alloy of iron, nickel, or cobalt, and/or a rare earth element or alloy such as gadolinium, neodymium, and/or holmium. For instance, the ferromagnetic components described herein may be comprised of any suitable material, and, in some examples, the ferromagnetic components may be made of a plurality of different materials, where the materials define different saturation levels. Although the examples of some of the treatment coils are described with the inclusion of a ferromagnetic component(s), any of the treatment coils described herein may include a subset of the illustrated ferromagnetic component(s) and/or the ferromagnetic component(s) may be omitted.

Also, it should be appreciated that a direct current (DC) magnetic field and/or permanent magnet may be used in any of the treatment coils described herein, for example, to modify the susceptibility of the ferromagnetic component to reshape the magnetic field and/or alter the separation of the activation zones created by the treatment coil.

Finally, it should be appreciated that any of the treatment coils described herein (may be configured to generate a magnetic field that induces one or more activation zones (e.g., two activation zones) within a subject (e.g., at one or more target locations). Accordingly, although the treatment coils are primarily described as generating a magnetic field that induces two activation zones, any of the treatment coils described herein may be configured to generate a magnetic field that induces more or less than two activation zones.

A patient may undergo TMS treatment over a period of several weeks or months. Throughout this period, the patient may receive treatment several days a week and often many days consecutively. TMS treatment may occur in multiple sessions on a single day.

As noted above, prior to treatment, the user's treatment location may first be determined. For TMS, for example, the treatment location is typically determined based on the subject's MT position, which itself is determined by moving the coil near a predicted area determined by patient anatomical landmarks until the desired motor response (e.g., thumb twitch) is achieved. The MT position is marked, for example, with an ink mark on the subject's head. In the case of using the TMS coil for treatment of depression, for example, the TMS therapy position is determined by moving the coil from the MT position along a line in the anterior direction a prescribed and/or predetermined distance (e.g., a distance ranging from 4.0 cm to 6.0 cm) to identify the treatment location on the subject.

Failure to properly position and orient the TMS apparatus at the treatment location may cause the treatment to be unsuccessful and/or create undesirable side effects. Therefore, it is important to ensure that the treatment coil is located at the same treatment location for every treatment. Conducting so many treatment sessions may cause inconsistent measurement of the treatment location, and, in turn, may lead to the incorrect neurons being depolarized and/or stimulated (e.g., neurons not associated with the treatment location). Incorrect positioning and/or orientation of the TMS apparatus for even a single treatment may lead to long-term and/or unintended adverse reactions.

Other factors may also lead to variances in MT position and treatment location measurements from treatment to treatment. For example, a patient may receive treatment at multiple different TMS treatment facilities and/or via different TMS coil apparatuses within the same facility. Each chair the patient sits in to receive TMS treatment may have its own height, rotation, and other positioning settings. Different physicians and/or technicians may interpret the initial MT measurements, and/or the measurements taken of the correct position and/or orientation settings of the TMS apparatus differently. No two physicians or technicians may apply the TMS apparatus to the patient's head in the same manner. Therefore, user inconsistencies, let alone user error, may lead to incorrect MT position measurement and improper placement of the TMS apparatus.

Additional factors that may lead to inconsistent MT measurements may include variance in positioning and/or orientation of TMS apparatuses with a different number of coils. For example, a single-coil TMS apparatus may have different shape and/or size than apparatus with two or more coils. Therefore, the MT position may change when using an TMS apparatus with spatial dimensions different than the TMS apparatus used in a previous treatment. A patient may treat with a single-coil apparatus on one day, return to the same treatment facility the following day, and then receive treatment with a TMS apparatus with multiple electromagnetic coils. A physician or technician may fail to account for changes in TMS apparatus shape, size, and coil number, which may inadvertently affect the MT position. Each of these variances, either alone or collectively, may cause the treatment coil to apply the electromagnetic field pulse to an area of the patient's brain other than vital treatment location. Accordingly, removal of these measurement variances as well as the factors which may lead to the measurement variances are critical to proper treatment.

Described herein is a head support guide system worn on the patient's head that ensures the TMS apparatus may be placed at the same position and orientation for every wear. The head support guide functions such that measurements for obtaining the MT position need only be performed on the initial TMS treatment and that measurements do not need to be repeated with every subsequent TMS treatment.

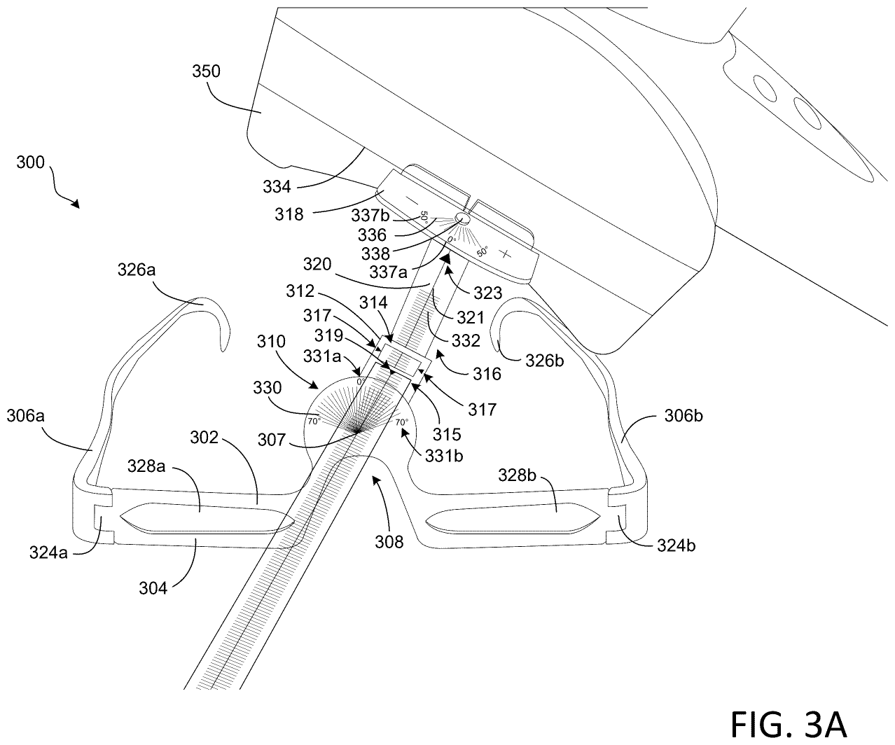

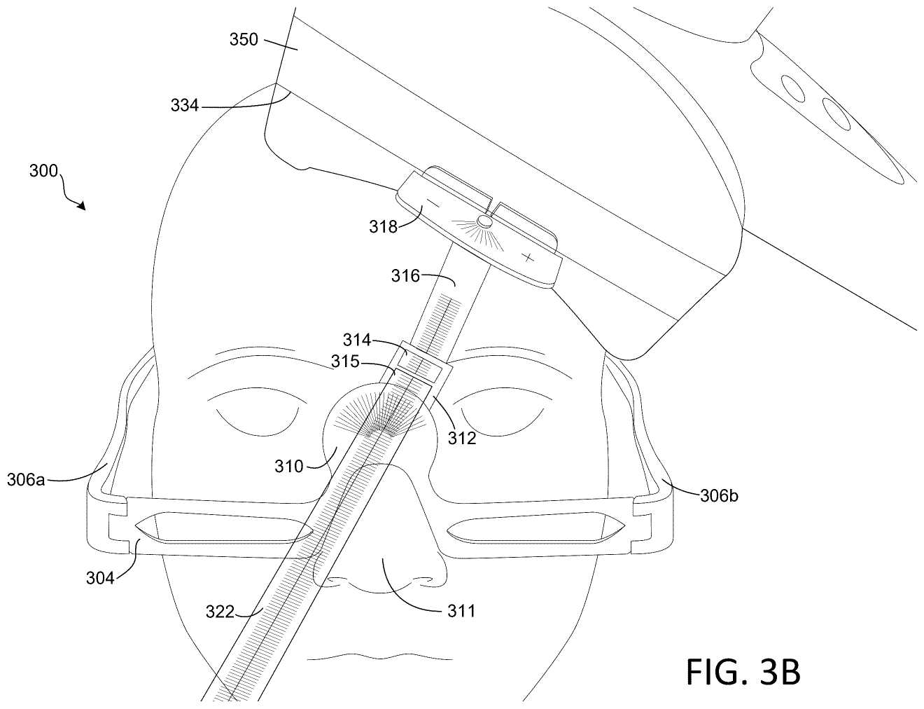

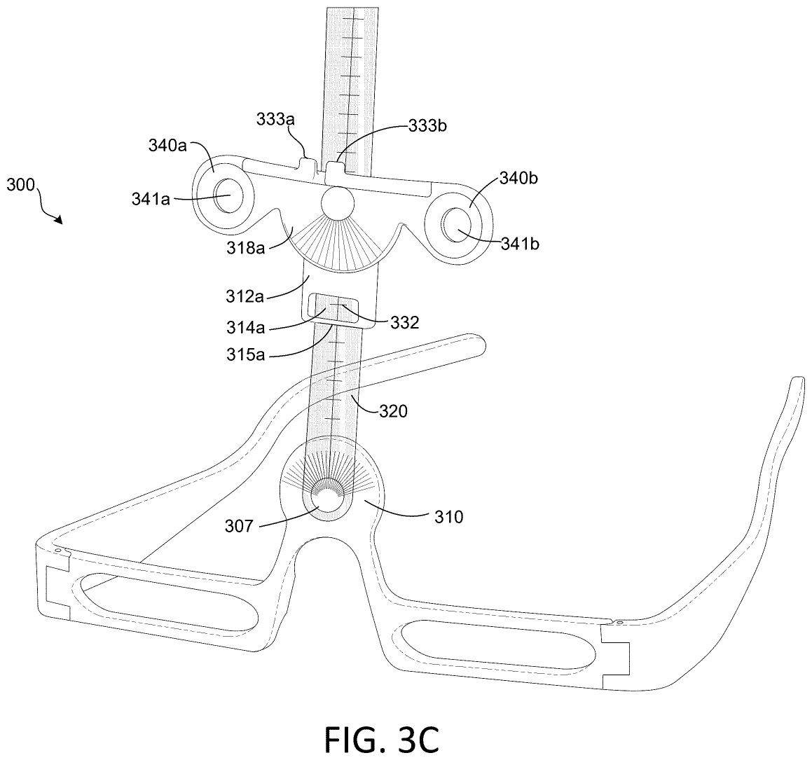

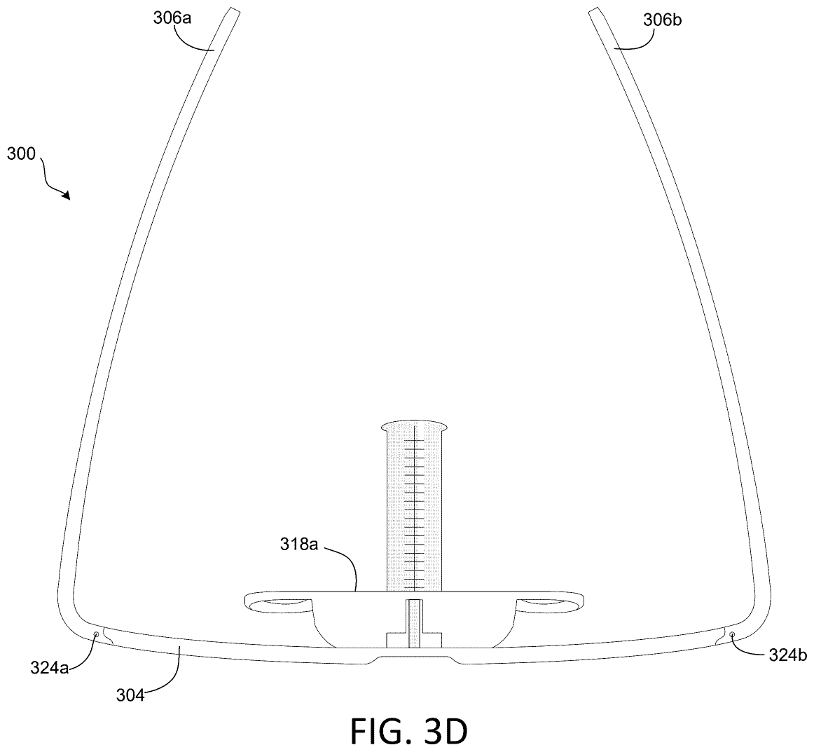

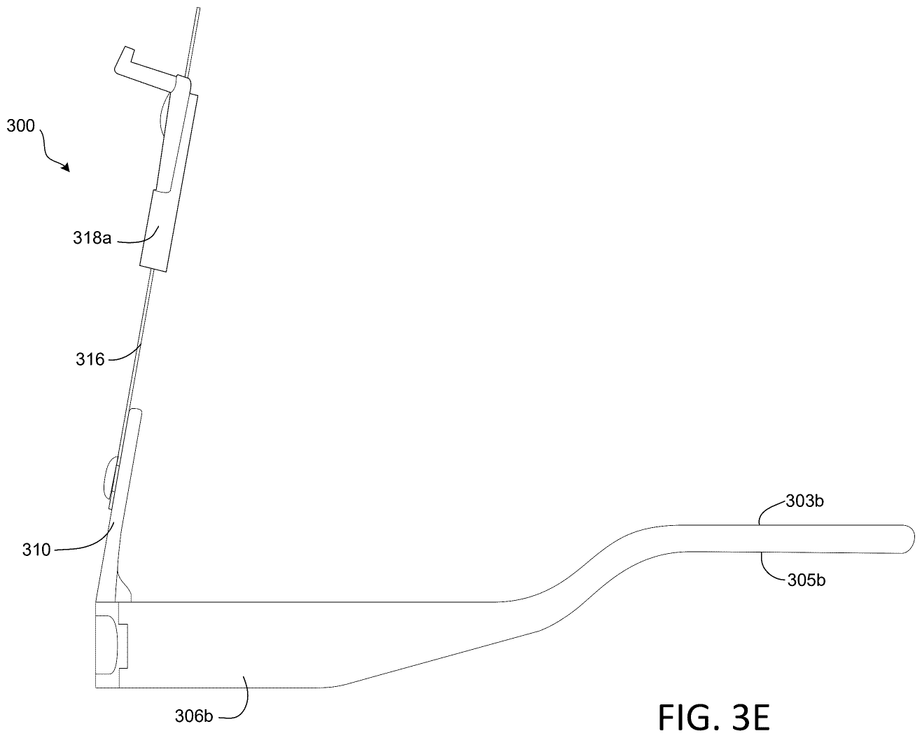

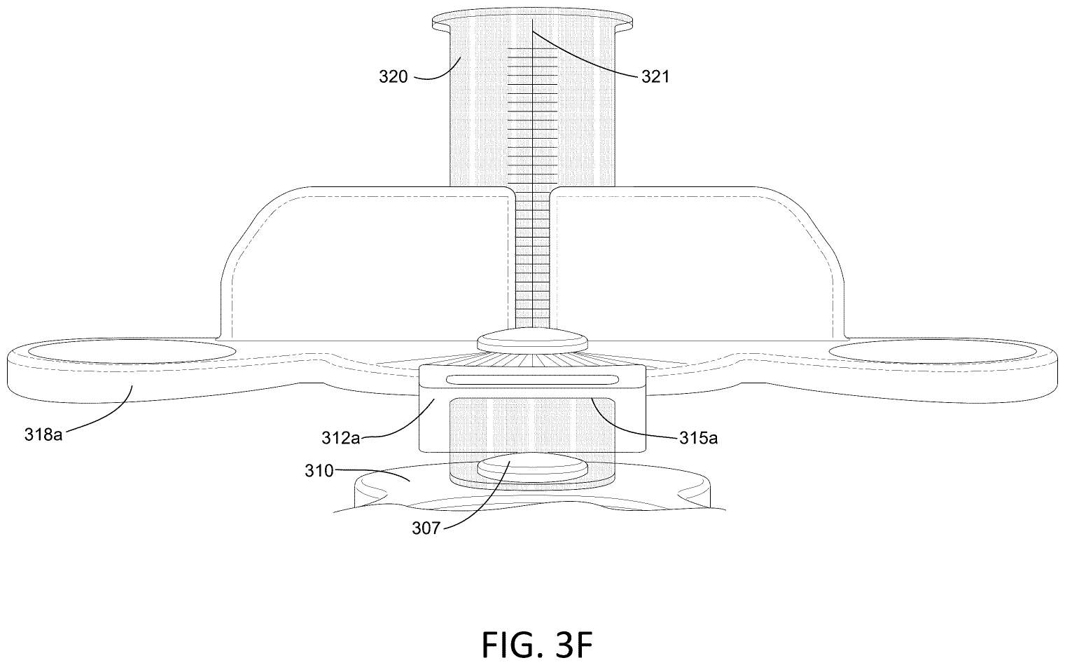

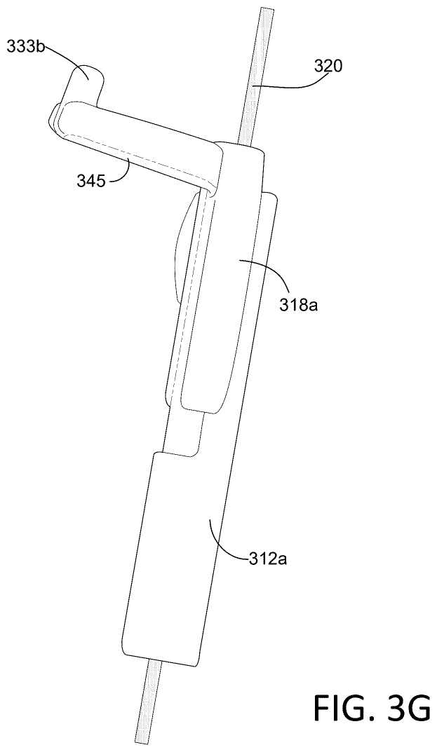

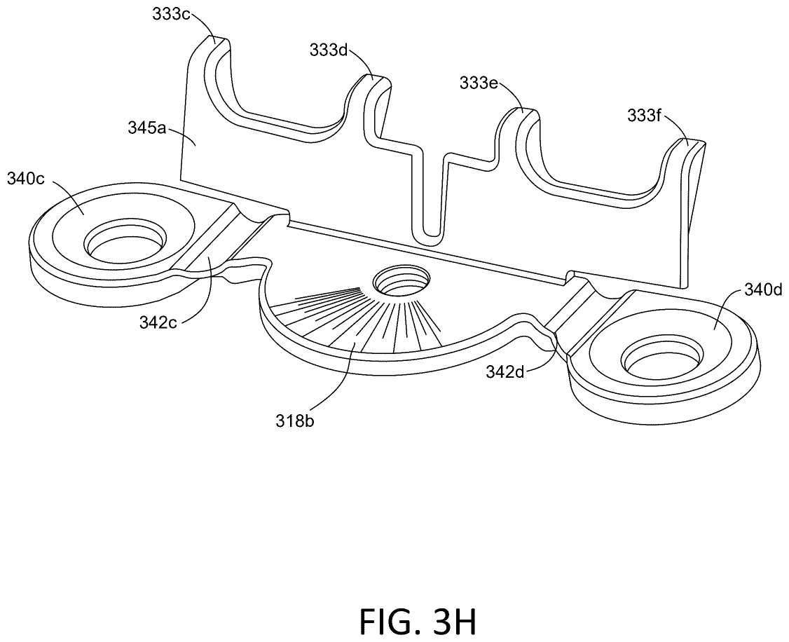

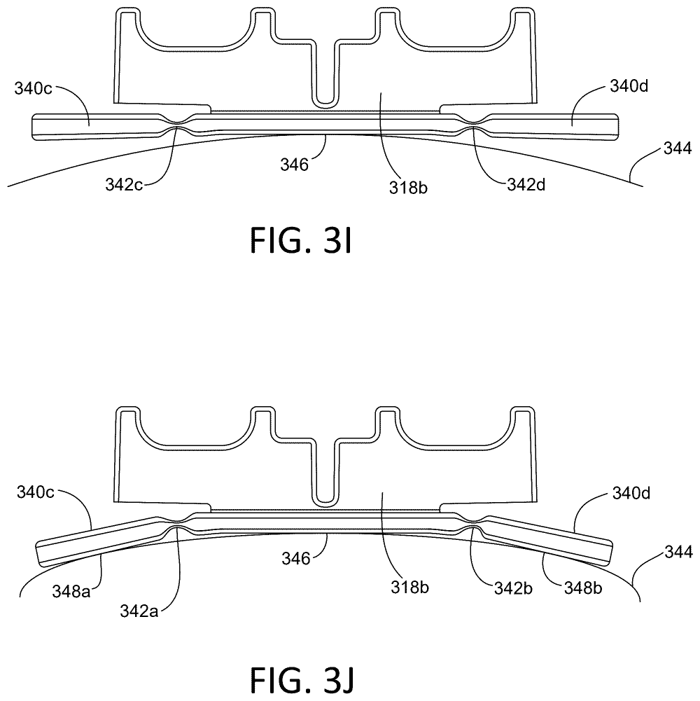

FIG. 3A is a diagram of an example head support guide 300 in contact with a treatment coil 350. The treatment coil 350 may be an example of the treatment coil 102 of FIG. 1 or the magnetic stimulation component 250 of FIG. 2. The head support guide 300 may be configured to measure the distance, position angle, and/or the orientation angle of the treatment coil 350 when the treatment coil 350 is located at the treatment location for the patient (e.g., and/or located at the MT position of the patient). For example, a technician may use the head support guide 300 after the treatment location of the patient is determined during an initial configuration for a treatment procedure. Thereafter, during subsequent treatments, the head support guide 300 may be used to place the treatment coil 350 at the treatment location (e.g., based on the measured distance, position angle, and/or the orientation angle), for example, without requiring the technician to replicate the entire MT detection and/or treatment location detection process that is required prior to the first treatment.

Moreover, these configuration techniques may be useful in neuronavigation. In neuronavigation, when observing a medical image, distance and/or direction to an anatomical and/or functional target may be measured relative to one or more landmarks. The treatment location may be found on a medical image after using the head support guide 300 and, for example, with or without use of a vision system, electromagnetic tracker, and/or other traditional neuronavigation device. A physician and/or technician observing a patient's brain in image(s) generated by a medical device (e.g., MRI) may apply the measurements taken during the treatment process to assist in observation and/or treatment. The generated images may be adapted to account for treatment coil geometries.

The head support guide 300 may include a support structure 302 that is configured to rest on a patient's head. In some examples, the support structure 302 may resemble a pair of eyeglasses, as depicted in FIG. 3A (e.g., without lenses). The support structure 302 may include an elongated crossbar 304 situated horizontally across the patient's face. The support structure 302 may be configured to rest on the patient's head like a pair of eyeglasses, or the support structure may rest below the patient's eyes of lower around the eye so as to avoid contact with the adjustable treatment coil 350. Located at the center of the crossbar 304 may be a notch 308 that is configured to rest on the patient's nose to support the support structure 302, (e.g., the bridge of a pair of eyeglasses). In some examples, the notch 308 may be of various dimensions and/or be adjustable to accommodate receiving different nose shapes. The sections of the crossbar 304 between notch 308 and the ends of the crossbar (e.g., where the crossbar 304 meets the arm bars 306a, 306b) may include a plurality of open windows, such as a first open window 328a and a second open window 328b (e.g., as depicted in FIG. 3A), resembling open frames of a pair of eyeglasses. The first and second open windows 328a, 328b may be configured with lenses, thereby enabling and/or improving the patient's vision while the patient wears the head support guide. Additionally or alternatively, sections of the crossbar 304 between notch 308 and the ends of the crossbar may be comprised of a solid and/or flexible piece of material.

Attached to each end of the crossbar 304 may be a first elongated arm bar 306a and a second elongated arm bar 306b. Each arm bar 306a, 306b may be an elongated element (e.g., eyeglasses temple) that is configured to attach to the crossbar 304 at one end and rest on the side of the patient's face. The first arm bar 306a may be attached to the crossbar 304 by a hinge 324a, and the second arm bar 306b may be attached to the crossbar 304 by a hinge 324b. Alternatively or additionally, the arm bars 306a, 306b may be attached to the crossbar 304 using a screw, brad, pin, and/or other hardware. The distal end of the arm bar 306a may define a curved and/or elongated extension 326a that is configured to wrap around the patient's ear for added stability (e.g., eyeglasses temples tips). Similarly, the distal end of the arm bar 306b may define a curved and/or elongated extension 326b that is configured to wrap around the patient's ear for added stability. In some examples, the arm bars 306a, 306b and the crossbar 304 may be formed by a single, continuous strip of material. The crossbar 304 and/or arm bars 306a, 306b may be made of a flexible material such that any portion and/or region of the strip may be bent, creased, curved, or adjusted so that the arm bars 306a, 306b wrap around the patient's head. In any configuration, the support structure 302 should be configured to remain stationary when the patient wears the head support guide 300.

The head support guide 300 may include any combination of different measurement instruments. For example, the head support guide 300 may include a first instrument, such as a position angle measurement instrument 310, that is configured to indicate a position angle for placement of the treatment coil 350. The head support guide 300 may include a second instrument, such as a linear measurement instrument 316, that is configured to indicate a distance from the support structure 302 for placement of the treatment coil 350. The head support guide 300 may include a third instrument, such as an orientation angle measurement instrument 318, that is configured to indicate an orientation angle for rotation of the treatment coil 350. Accordingly, the head support guide 300 may be configured to measure the distance, position angle, and/or the orientation angle of the treatment coil 350 when the treatment coil 350 is located at the treatment location for the patient (e.g., and/or located at the MT position of the patient).

The position angle measurement instrument 310 may be configured to measure the position angle of the treatment coil 350 with respect to the median plane of the patient. The median plane, or the mid-sagittal plane as it is also commonly known, refers to a plane that vertically bisects the human body through a midline that passes through the area between the eyes, the middle of the nose, the chest, and navel. In other words, the median plane evenly divides the human body into left and right halves. The position angle measurement instrument 310 may be configured to indicate the position angle for placement of the treatment coil 350, for example, when the treatment coil 350 is located at the treatment location for the patient. One example of the position angle measurement instrument 310 is a protractor.

The position angle measurement instrument 310 may be connected to the support structure 302 or integrated with the crossbar 304. For example, the position angle measurement instrument 310 may be located in the center of the crossbar 304, immediately above the notch 308. For example, a position origin 307 (e.g., center point) of the position angle measurement instrument 310 may be located in the center of the crossbar 304. Accordingly, the position angle measurement instrument 310 may be integrated with or attached to the support structure 302, and as such, the position angle measurement instrument 310 may remain stationary when the head support guide 300 remains in use. Therefore, the movement of the position angle measurement instrument 310 is restricted. The position angle measurement instrument 310 may be configured such that it does not rotate or shift when the treatment coil 350 changes position or orientation on the patient's head. The position angle measurement instrument 310 may be comprised of the same material as the crossbar 304 and/or arm bars 306a, 306b.

The position angle measurement instrument 310 may include indicia, such as position angle lines 330 that denote different angle measurements (e.g., degree markers). In other examples, a rotary encoder may measure the position angle and output the measurement reading via an electronic angle display. In some examples, such as that depicted in FIG. 3A, the position angle lines 330 may range in value from 0° to 70°. In other examples, the position angle lines may range in value from −90° to 90°. Angle measurements may have specific numbers (e.g., 0° and 70°) drawn or printed on the position angle measurement instrument 310 near the appropriate position angle line 330. For example, in FIG. 3A, these position angle lines 330 measuring 0° and 70° may be seen at 331a and 331b, respectively. A measurement of 0° may be at the top, center of the position angle lines and oriented perpendicularly relative to the support structure 302. Although illustrated as position angle lines 330, in some examples, the position angle measurement instrument 310 may include other indicia, such as notches, markings, and/or any indicator that indicate the different angle measurements. And, instead of degree markers, the indicia (e.g., position angle lines 330), may further include one or more numbers, letters, and/or symbols.

As noted above, the head support guide 300 may include the linear measurement instrument 316, which may be configured to measure linear distance (e.g., using a ruler or tape measure). For example, the linear measurement instrument 316 may be configured to measure the distance between the support structure 302 and the treatment coil 350. The linear measurement instrument 316 may include strip 320 that may operate as a ruler, tape measure, or linear encoder. The strip 320 may be made of a thin, flexible material such that any portion and/or region of the strip may be bent, creased, curved, or adjusted. The strip 320 may feature a center line 321 that runs parallel to the elongated edges of the strip. A center line 321 may be printed and run down the center of the strip 320 such that the strip 320 is bisected into two elongated, narrow halves of equal area. On the center line 321 may be a center line indicia 323 that aligns with the center line 321. The center line indicia 323 may be printed and/or drawn at any point on the center line 321. The center line indicia 323 may be a triangle (as depicted in FIG. 3A), arrow, or other marking that helps delineate the center line 321. As seen in FIG. 3A, the center line indicia 323 may be shaped like a triangle and printed and/or drawn the end of the strip 320 substantially adjacent to the treatment coil 350. The strip 320 may include additional indicia, such as lines 332 that denote measurements along the strip 320. As depicted in FIG. 3A, the lines may be perpendicular to the center line 321 and long edges of the strip 320. To ensure proper measurement accuracy, the distances between each indicia (e.g., line 332) may be equal. Although illustrated as lines 332 in FIG. 3A, in other examples the strip 320 may include other indicia, such as notches, markings, and/or any indicator. The indicia may further include one or more numbers, letters, and/or symbols.

One end of the linear measurement instrument 316 may be coupled with the support structure 302 and/or the position angle measurement instrument 310. For example, the linear measurement instrument 316 may include a position measurement indicator 312. The position measurement indicator 312 may be affixed (e.g., rotationally affixed) to the position angle measurement instrument 310 such that the position measurement indicator 312 may be free to rotate around the position angle measurement instrument 310. For example, the position measurement indicator 312 may be affixed (e.g., rotationally affixed) to the position origin 307 of the position angle measurement instrument 310 and/or to the center point of the crossbar 304. The position measurement indicator 312 may be attached to the position angle measurement instrument 310 and/or the crossbar 304 by a screw, brad, pin, and/or other hardware that permits the position measurement indicator 312 to freely rotate about the position angle measurement instrument 310. The position measurement indicator 312 may be affixed (e.g., rotationally affixed) to either the front side of the position angle measurement instrument 310 (e.g., where the indicia for measuring the position angle, such as the position angle lines 330, are featured), or the reverse side of the position angle measurement instrument 310, for better measurement readings of the position angle (as depicted in FIG. 3A). The position angle instrument 310 may be configured with rotational range of motion with friction fit or drag. This configuration may allow the position angle instrument 310 and/or any elements affixed to the position angle instrument 310 to maintain rotational range of motion yet move over the range but hold their position.

The position measurement indicator 312 may include a top plate and/or a bottom plate (not shown in FIG. 3A). The bottom plate and top plate may be two distinct pieces configured to clasp, interlock, or otherwise close and/or press together. Additionally or alternatively, the top and bottom plates may be connected by a hinge and/or other connector that allows the plates to open without separating from each other. When the top and bottom plates are connected together, the position measurement indicator 312 may include an opening at its distal end. This opening may be configured to receive the strip 320, such that the strip 320 may enter into an inner cavity of the position measurement indicator 312 (e.g., between a top and bottom plate of the position measurement indicator 312) at the distal end.

The position measurement indicator 312 may include one or more openings, windows, slots, and/or similar aperture. For example, the position measurement indicator 312 may include a first slot 314 and a second slot 315. After passing through the opening at the distal end of the 312, the strip 320 may pass by the first slot 314 and then exit the inner cavity of the position measurement indicator 312 through the second slot 315. When passing through the first slot 314, the lines 332 (or other indicia that may denote measurements along the strip 320) may be easily viewed through the opening (e.g. viewing window) provided by the first slot 314.

When extending through the position measurement indicator 312 and exiting through the second slot 315, the strip 320 may be held within place. The strip 320 may be configured to extend into and out of the position measurement indicator 312 based on the relative position of the treatment coil 350. For example, if the treatment coil 350 shifts in the posterior direction (e.g. away from the head support device 300), the linear measurement instrument 316 will shift with it, thereby forcing more of the strip 320 to be pulled back through the opening of the position measurement indicator 312. Any length of strip 320 that exits out of the second slot 315 may be placed (e.g., draped) over the position angle measurement instrument 310. Additionally or alternatively, any length of strip 320 that exits out of the second slot 315 may be placed (e.g., draped) behind the support structure 302.

The position measurement indicator 312 may include distance indicia 317 found adjacent the first slot 314. These distance indicia 317 may be used to indicate the linear distance between the support structure 302 and the treatment coil 350, for example, when the treatment coil 350 is located at the treatment location. The distance indicia 317 may be one or more triangles (as depicted in FIG. 3A), arrows, or other markings that guide the user to better identify the linear distance measurement. The distance indicia 317 may be printed and/or drawn on position measurement indicator 312 substantially adjacent to, and pointing at and/or towards, the lines 332 showing through the first slot 314.

The distance between the support structure 302 and the treatment coil 350 may be denoted by whichever line(s) 332 align with the distance indicia 317. As described above, the opening (e.g. viewing window) provided the first slot 314 may provide the technician with a clear view of the line(s) 332 that aligns with the distance indicia 317.

The position measurement indicator 312 may also include angle indicia 319. As described above, the position measurement indicator 312 may be configured to rotate about the position angle measurement instrument 310 and/or the center of the crossbar 304. The angle indicia 319 may indicate the position angle on the position angle measurement instrument 310, for example, when the treatment coil 350 is located at the treatment location. The angle indicia 319 may be a triangle (as depicted in FIG. 3A), arrow, or other marking drawn that guide the user to better identify the position angle. As seen in FIG. 3A, the angle indicia 319 may be printed and/or drawn position measurement indicator 312 substantially adjacent to, and pointing at and/or towards, the position angle measurement instrument 310.

The strip 320 may be transparent, such that the angle indicia 319 and/or line(s) 332 are visible through the strip 320. The angle indicia 319 may identify (e.g., point at and/or towards) the position angle line(s) 330 located on the position angle measurement instrument 310, such that the identified position angle line(s) 330 may be the current position angle of the treatment coil 350. As noted above, the head support guide 300 may also include the orientation angle measurement instrument 318. The orientation angle measurement instrument 318 may be configured to measure an orientation angle of the treatment coil 350, for example, when the treatment coil 350 is located at the patient's treatment location. In some examples, and as depicted in FIG. 3A, the orientation angle measurement instrument 318 may be coupled to a distal end of the linear measurement instrument 316.

The orientation angle measurement instrument 318 may be a protractor configured to measure an angle of the treatment coil 350 that denotes the treatment coil's orientation relative to the support structure 302. More specifically, as described in more detail below, the orientation angle measurement instrument 318 may measure the angle created by the orientation of the treatment coil 350 on the patient's head with respect to the line formed by the MT position and a reference point on the patient's face and the line formed by the MT position and edge of the treatment coil 350. In other examples, a rotary encoder may measure the orientation angle and output the measurement reading via an electronic display.

The orientation angle measurement instrument 318 may contact and/or affix with the front edge 334 of the treatment coil 350 and configured such that it moves contemporaneously and in the same directions and/or rotates with the treatment coil 350. The orientation angle measurement instrument 318 may also detach from the treatment coil 350 and/or may be repositioned elsewhere on the front edge 334. As depicted in FIG. 3A, the orientation angle measurement instrument 318 may be positioned at the center of the front edge 334 of the treatment coil 350. However, in examples, the orientation angle measurement instrument 318 may contact and/or be affixed to any point along the front edge 334 of the treatment coil 350. The orientation angle measurement instrument 318 may also be affixed to the position angle measurement instrument 310 and/or strip 320. The orientation angle measurement instrument may be affixed to the position angle measurement instrument 310 and/or strip 320 while contemporaneously affixed to the coil 350. The orientation angle measurement instrument may be coupled with a retraction mechanism (not pictured in FIG. 3A) that may guide the coil 350 into proper place over the MT position. In examples, the coil 350 may provide a signal (e.g., an electronic feedback signal) to a device on the head support guide system 300 that confirms that the coil 350 is properly aligned on the patient's head (e.g., the coil 350 is in the proper position to perform TMS treatment on the treatment location).

The orientation angle measurement instrument 318 may include indicia, such as orientation angle lines 336 that denote different angle measurements (e.g., degree markers). In examples, in addition to orientation angle lines 336, the indicia may be notches, markings, and/or any indicator that denotes different angle measurements. As depicted in FIG. 3A, the orientation angle lines 336 may range in value from 0° to 50°. In examples, the orientation angle lines 336 may range in value from −90° to 90°. Angle measurements (e.g., 0° and 50°) may have specific numbers drawn or printed on the orientation angle measurement instrument 318 near the appropriate orientation angle line 336. For example, in FIG. 3A, these orientation angle lines 336 measuring 0° and 50° may be seen at 337a and 337b, respectively. In examples, and as depicted in FIG. 3A, the orientation angle measurement instrument 318 may be configured such that an orientation angle line 336 drawn where the orientation angle measurement instrument 318 is perpendicular (e.g., reads 0°) with the front edge 334 of the treatment coil 350. When in this position, the orientation angle line 336 that reads 0° aligns with the center line 321 and center line indicia 323 of the strip 320.

The distal end of the linear measurement instrument 316 may be affixed (e.g., rotationally affixed) to the orientation angle measurement instrument 318 such that the orientation angle measurement instrument 318 may be free to rotate about the linear measurement instrument 316. When the linear measurement instrument 316 is oriented perpendicular to the treatment coil 350, the orientation angle measurement instrument 318 may read 0°. An orientation origin 338 (e.g., or center point) may be located on the orientation angle measurement instrument 318. This orientation origin 338 may be where the linear measurement instrument 316 affixes (e.g., rotationally affixes) to the orientation angle measurement instrument 318. The linear measurement instrument 316 may affix (e.g., rotationally affix) to the orientation angle measurement instrument 318 at the orientation origin 338 by a screw, pin, brad, and/or other hardware that permits the linear measurement instrument 316 to rotate about the orientation angle measurement instrument 318.

FIG. 3B depicts an example head support guide 300 in contact with the treatment coil 350 and being worn by a patient. FIG. 3B details the primary components of the head support guide 300 as configured around the patient's head. For example, crossbar 304 may rest on the patient's nose 311, with the patient's nose occupying the space provided by notch 308 (not pictured in FIG. 3B). In other words, when the head support guide 300 is not worn by the patient, the notch 308 may be a vacant space. The position angle measurement instrument 310, configured on the crossbar 304 (as pictured in FIG. 3B), may rest immediately above the patient's nose. First and second arm bars 306a, 306b each may extend to and rest on one of the patient's ears.

As depicted in FIG. 3B, the treatment coil 350 may be in position over the treatment location. In this configuration, orientation angle measurement instrument 318 may be attached to the front edge 334 of treatment coil 350. Coupled to the orientation angle measurement instrument 318 is the linear measurement instrument 316. The strip 320 of the linear measurement instrument 316 may extend into the position measurement indicator 312, through the window of the first slot 314, and may exit out of the second slot 315. The excess section of strip 320 that exits out of second slot 315 may be featured in FIG. 3B as being draped over the position angle measurement instrument 310 and further extend over the part of the patient's nose. In examples, the strip 320, which is made of a transparent material, allows the technician to read lines 332 on the position angle measurement instrument 310. It should be noted that in FIG. 3B, the position measurement indicator 312 is affixed (e.g., rotationally affixed) to the back of position angle measurement instrument 310.

FIG. 3C depicts a front perspective view of the head support guide 300 when not worn by the patient. FIG. 3C further depicts an example alternative orientation angle measurement instrument 318a as decoupled and/or disconnected from the treatment coil 350 (not pictured in FIG. 3C). For instance, although similar reference numbers are used, the relative orientation and/or position of certain aspects of the head support guide 300 (e.g., the orientation angle measurement instrument 318a) may be altered.

Position indicia on the strip 320, such as lines 332, may again be viewed through a first slot 314a. The strip 320 may exit a second slot 315a, for example, as described above before passing over the position angle measurement instrument 310 and terminating at the position origin 307. In this configuration, a position measurement indicator 312a is affixed to the orientation angle measurement instrument 318a, as opposed to being affixed (e.g., rotationally affixed) to the of the position angle measurement instrument 310 as depicted in FIGS. 3A and 3B. In other words, this alternative form of the orientation angle measurement instrument 318a may form a single, contiguous component with the position measurement indicator 312a, as opposed to the separated components depicted in FIGS. 3A and 3B. This alternative form of the orientation angle measurement instrument 318a does not have the strip 320 exit out of the second slot 315a, unlike the orientation angle measurement instrument 318 depicted in FIGS. 3A and 3B, wherein the strip 320 exits out of the second slot 315. The orientation angle measurement instrument 318a and position measurement indicator 312a may be a single, continuous article. In examples, the orientation angle measurement instrument 318a and position measurement indicator 312a may be detachable, separable, and/or distinct elements as described above and depicted in FIGS. 3A and 3B. Moreover, in FIG. 3C, and in contrast to the previously described examples, the strip 320 may be affixed (e.g., rotationally affixed) to the front of the position angle measurement instrument 310 at position origin 307. The strip 320 may not extend past the position origin 307, and instead may terminate at the point of rotational connection to the position angle measurement instrument 310 (e.g., the position origin 307).

The ears 340a, 340b of the orientation angle measurement instrument 318a may be used by the physician and/or technician to move the orientation angle measurement instrument 318a into the desired position. The interior circumference of the ears 340a, 340b may be pivoted inward such that the ears 340a, 340b are better adapted for fingertips. The center of the ears 340a, 340b may include open windows, such as a first open window 341a and a second open window 341b.

FIG. 3C further features adapter tabs 333a, 333b, which protrude from the top of orientation angle measurement instrument 318a. These adapter tabs 333a, 333b may be used to attach the orientation angle measurement instrument 318a to treatment coil 350, or another device for added stability, and/or measurement quality.

FIG. 3D depicts a top-down plan view of the head support guide 300 when not worn by the patient. FIG. 3D shows that the first and second arm bars 306a, 306b each may attach to an opposite end of crossbar 304 at hinge 324a and 324b, respectively. From this perspective, the crossbar may be curved (e.g., bowed) such that each end may better adapt to (e.g., be more secure) a patient's head. Similarly, the first and second arm bars 306a, 306b may not be affixed at the hinges 324a, 324b such that they form right angles with the crossbar 304. Rather, the distal ends of the first and second arm bars 306a, 306b may angle inwards to better wrap (e.g., adapt to and/or secure) around patient's head.

FIG. 3E depicts a right side view of the head support guide 300. In this example, first (not pictured in FIG. 3E) and second arm bar 306a, 306b have a crooked design, such that the top edge 303a, 303b, respectively, and bottom edge 305a, 305b, respectively, of the first and second arm bar 306a, 306b may not be straight lines (top edge 303a and bottom edge 305a of first arm bar 306a are not shown in FIG. 3E). FIG. 3E also illustrates a side view of position angle measurement instrument 310, linear measurement instrument 316, and the orientation angle measurement instrument 318a as depicted in FIG. 3C, all coupled together as described above. FIG. 3E further illustrates how the position angle measurement instrument 310, linear measurement instrument 316, and orientation angle measurement instrument 318a, from the depicted side view, may not be connected to the cross bar 304 at a right angle. Rather, the instruments may be angled toward the direction of the patient's face to better match the anatomical taper of the patient's head.

FIG. 3F provides a detailed view, from the bottom/front perspective, of the position angle measurement instrument 310, linear measurement instrument 316, and orientation angle measurement instrument 318a as depicted in FIGS. 3C and 3E. The distal end of strip 320 may originate from a part of the head support guide 300 that extends beyond the treatment coil 350 (not pictured in FIG. 3F). In this example, the strip 320 extends behind the orientation angle measurement instrument 318a. The strip 320 extends through the position measurement indicator 312a, and exits through position measurement indicator 312a at the second slot 315a. FIG. 3F further depicts, for example, the strip 320 terminating at the point of rotational connection to the position angle measurement instrument 310 (e.g. the position origin 307). The center line 321 runs down the center of the strip 320 to ensure that the strip is properly aligned and evenly inserted into the position measurement indicator 312a.

FIG. 3G depicts a right side view of the alternative version of orientation angle measurement instrument 318a as depicted in FIGS. 3C, 3E, and 3F. Strip 320 may be fed through the center of the orientation angle measurement instrument 318a and exit through the second slot 315a of position measurement indicator 312a. As described above, the position measurement indicator 312a may be the orientation angle measurement instrument 318a and position measurement indicator 312a may be detachable, separable, and/or distinct elements or a single, continuous article as depicted in FIG. 3G. Further depicted are the adapter tabs 333b (adapter tab 333a not shown in FIG. 3G) which may be configured with additional measurement instruments and/or articles to better secure the head support guide 300 to the treatment coil 350 (not pictured in FIG. 3G). Adapter tab 333b (and/or, adapter tab 333a not shown, and any additional adapter tabs not shown) protrudes from the back wall 345 of the orientation angle measurement instrument 318a, wherein the back wall 345 extends substantially perpendicularly from the part of the orientation angle measurement instrument 318a featuring the orientation angle lines 336.

FIG. 3H depicts a front perspective view of another alternative orientation angle instrument 318b. Extending from a back wall 345a of this orientation angle instrument 318b may be one or more adapter tabs 333c, 333d, 333e, and 333f. The adapter tabs 333c, 333d, 333e, and 333f may be configured to better align with the contours of the treatment coil 350 for added stability. The back wall 345a and/or adapter tabs 333c, 333d, 333e, and 333f may be made of a flexible material to better account for the rotation and positioning of the treatment coil 350. The ears 340c, 340d may be affixed (e.g. flexibly affixed) to the rest of the orientation treatment angle instrument 318b at hinges 342c and 342d, respectively. The hinges 342c and 342d may bend such that the ears 340c, 340d may flap rotationally about the hinges 342c and 342d, respectively.

FIGS. 3I and 3J depict a front/top perspective view of the orientation angle instrument 318b introduced in FIG. 3H as it rests on the surface of a patient's head 344. FIG. 3I illustrates the orientation angle instrument 318b as it is initially placed on a patient's head. In FIG. 3I, the physician and/or technician has not conformed the orientation angle instrument 318b to the patient's skull. The ears 340d, 340d may be coplanar with the rest of orientation angle instrument 318b and have not yet made contact with the surface of the patient's head 344. In this configuration, only the orientation angle instrument 318b contacts the surface of the patient's head 344 at contact point 346. With no other points contacting the surface of the patient's head 344, the orientation angle instrument 318b may not be secure on the patient's head. Accordingly, the orientation angle instrument 318b may require additional fastening to obtain the proper measurements. When ready to take measurements and/or secure the orientation angle instrument 318b to the surface of the patient's head 344, the physician and/or technician may press the ears 340c, and 340d such that the ears 340c, 340d bend about the hinges 342c, 342d. The ears 340c, 340d may contact the surface of the patient's head 344 at ear contact points 348c, 348d, respectively. In this configuration, the ears 340c, 340d will no longer be coplanar with the rest of the orientation angle instrument 318b, but flex down to create a more stable resting position on the surface of the patient's head 344.

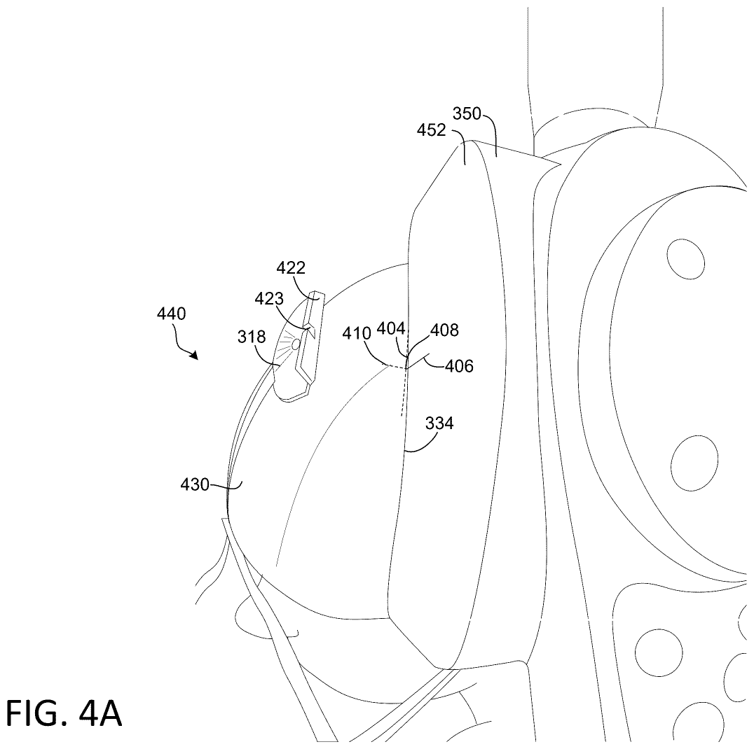

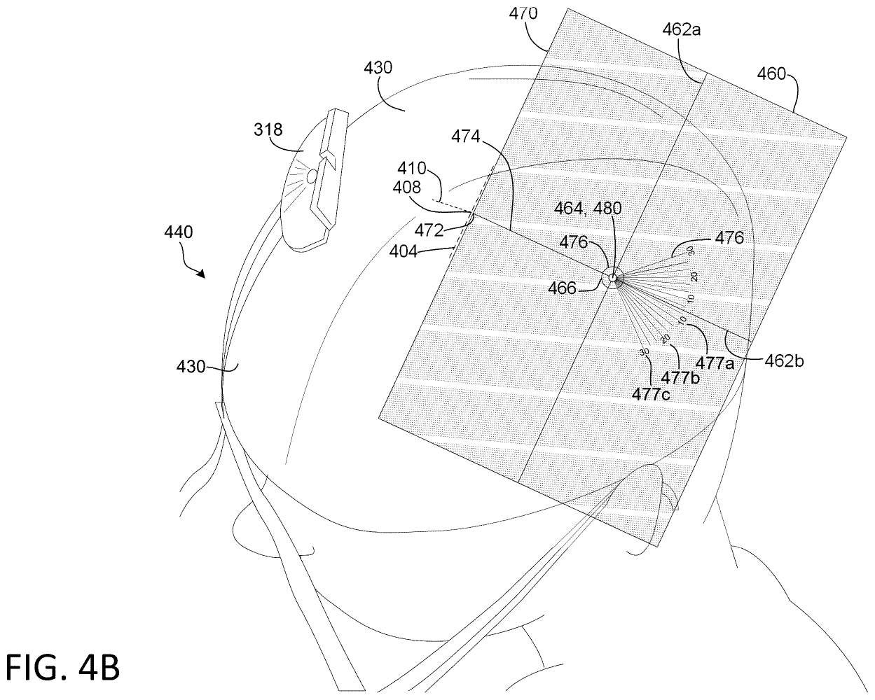

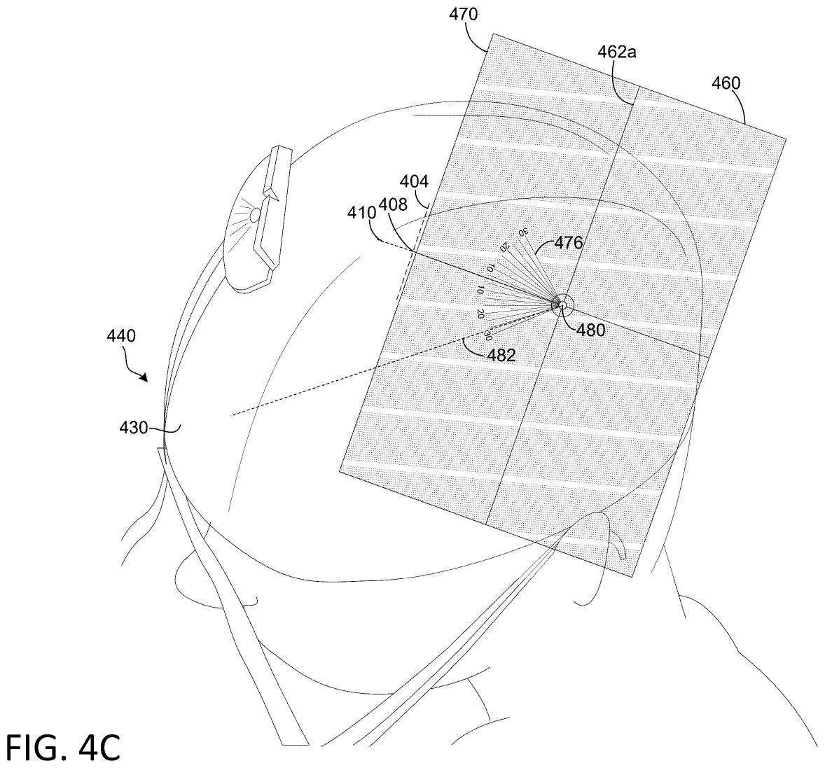

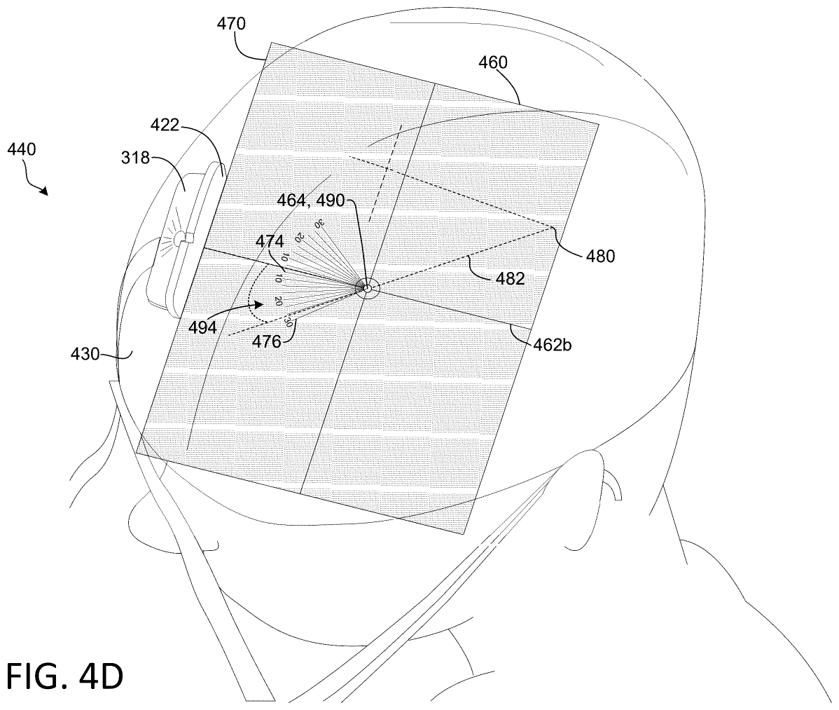

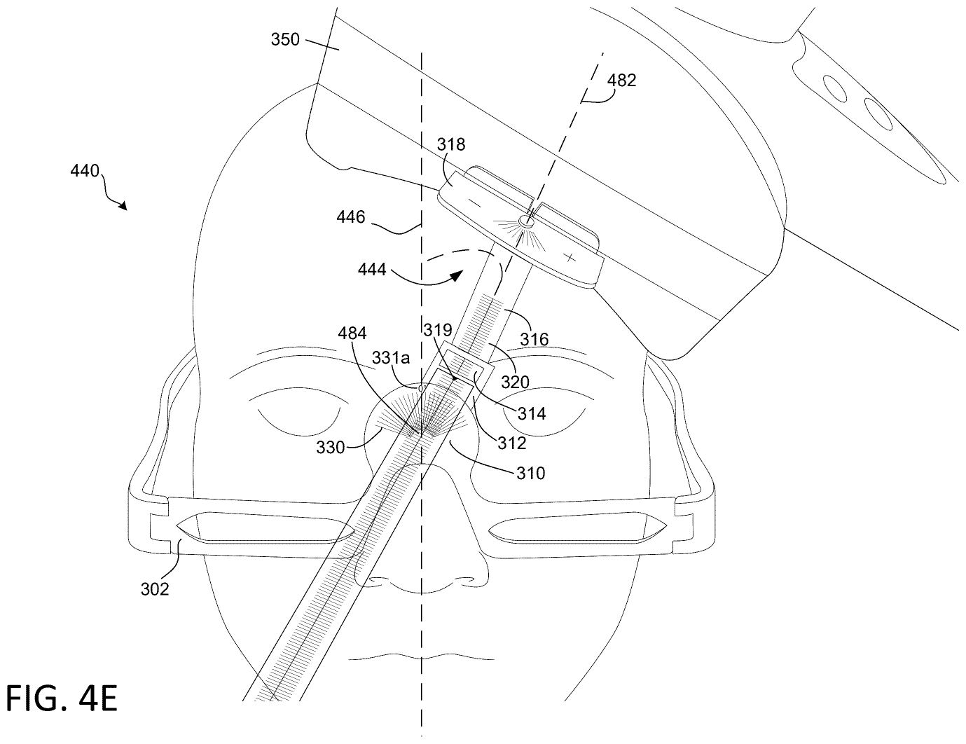

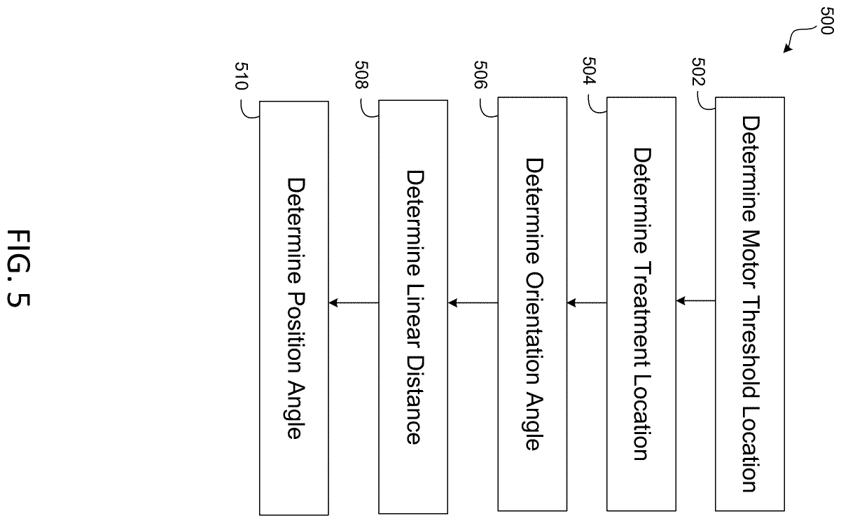

FIGS. 4A-D depict a process for obtaining the measurements the treatment location when the patient wears the head support guide system 300 during initial TMS treatment setup. The procedure described in reference to FIGS. 4A-D may be performed to calibrate the head support guide 300 during an initial visit by a patient 440. For instance, the technician may determine the measurement values of the measurement instruments of the head support guide 300 (e.g., the position angle measurement instrument 310, the linear measurement instrument 316, and the orientation angle measurement instrument 318), and thereafter. The technician may obtain these measurement values during subsequent visits and treatment procedures of the patient 440. The head support guide 300 and the determined measurements may be used to position the treatment coil 350 directly at the treatment location for the patient 440 without having to first identify the MT position of the patient and then determine the treatment location for the treatment coil 440. As such, the treatment procedure is benefited, for example, by reducing the overall time that the patient 440 has to sit in the chair, thereby reducing the likelihood that the treatment coil 440 is misoriented.

In FIG. 4A, the treatment coil 350 may be placed on the head of a human subject 440. The human subject 440 may wear a head covering 430 (e.g., a skull cap) worn to facilitate the recording of critical lines, points, measurements, and/or notations used in establishing the treatment location. In examples, the patient may wear any head covering that facilitates the recording of critical lines, points, measurements, and/or notations used in establishing the treatment location. The technician may write the measurements and/or markings on the head covering 430. The center of the treatment coil 350 is placed directly over a target location (e.g., the MT position, not pictured). As discussed above, the technician may first determine the MT position of the patient 440 prior to beginning the procedure described in reference to FIGS. 4A-D. For example, the MT position may be determined by moving the treatment coil 350 and achieving a desired motor response from the patient (e.g., thumb twitch). As also noted, performing the motor threshold procedure to obtain the MT position is a preliminary step completed prior to identifying the treatment location of the patient 440 and initiating the TMS treatment. As discussed above, the treatment location may be obtained by analyzing EEG coordinates, the beam F3 method, use of MRI testing, and/or from MNI coordinates in lieu of performing the motor threshold procedure. Further, in some examples, a secondary position may be determined relative to the first location (e.g., MT position) by changing the coordinates, for instance, using a calculation program (e.g., the calculation program could be used just to adjust the coordinates to rotate the coil in place).

With the treatment coil 350 located at the MT position of the patient 440, a technician may first mark on the head covering 430 the front edge 334 of the treatment coil 350. As depicted in FIG. 4A, the front edge 334 may be recorded as a dashed front edge line 404. The fixed docking point 406 (e.g., the center point) of the front edge 334 of the treatment coil 350 may be marked and/or printed on the treatment coil 350 with a solid line. The technician may mark the fixed docking point 406, or the fixed docking point 406 may be marked and/or printed on the treatment coil in advance of using the treatment coil 350. Where the front edge line 404 and center of the treatment coil 406 intersect may be recorded on the head covering 430 as edge point 408. To better identify where the fixed docking point 406 of the front edge 334 of the treatment coil 350 lies on the head covering 430, a perpendicular marking line 410 (depicted as a dashed line in FIG. 4A) may be recorded on the head covering 430. In other words, the perpendicular marking line 410 is oriented at a right angle from the front edge line 404. When the treatment coil 350 is removed from the patient's head, the location of the edge point 408 (e.g., where the fixed docking point 406 of the treatment coil 350 lies on the patient's head during treatment) may be permanently recorded on the head covering 430. Edge point 408 represents the intersection of the front edge line 404 and perpendicular marking line 410.

After determining and/or recording the front edge line 404, perpendicular marking line 410, and edge point 408, the treatment coil 350 may be removed. As depicted in FIG. 4B, after removal of the treatment coil 350, a measurement template 460 may be placed on the head covering 430. The measurement template 460 may be made of a thin, flexible material, but is not so pliable as to cause the measurement template 460 to bend, crease, curve, and/or otherwise warp. For example, the measurement template 460 may be made of plastic, cardboard, and/or any material that may adapt to the contours of the patient's head and still guide a technician in measuring and recording measurements. Though the measurement template 460 depicted in FIG. 4B is rectangular, it may be any shape that has straight lines as outer edges (e.g., a square, triangle, quadrilateral, etc.). The measurement template 460 may be transparent, such that the front edge line 404, edge point 408, perpendicular marking line 410, and any other markings, measurements, and/or notations recorded on the head covering 430 may be visible through the measurement template 460.

The measurement template 460 defines an edge 470. The technician may place the edge 470 of the measurement template 460 such that the edge 470 aligns along the front edge line 404. Traversing the entire length (e.g., the longer dimension) and entire width (e.g., the shorter dimension) of the template 460 are the length guideline 462a and the width guideline 462b, respectively. Length and width guidelines 462a, 462b may be printed on the measurement template 460 such that they bisect the measurement template 460 precisely in half in both length and width, respectively. Length and width guidelines 462a, 462b may intersect at the center of the measurement template 460 at right angles, where they define template center point 464. This template center point 464 may be surrounded by a circle 466 of minimal diameter. The circle 466 may further guide the user in making and/or recording the measurements described herein. Precisely at the template center point 464 is a hole that protrudes through the measurement template 460 such that the head covering 430 (e.g., or if no head covering is used, the patient's scalp, and/or hair), may be exposed through the hole.

Emanating from the template center point are a series of indicia, for example, angle measurement lines 476, that denote different angle measurements (e.g., degree markers). As depicted in FIG. 4A, the angle measurement lines 476 range in value from 0° to 30°. In examples, the angle measurement lines 476 may range in value from −90° to 90°. Angle measurements (e.g., 10°, 20°, and/or 30°) may have specific numbers drawn or printed on the measurement template 460 near the appropriate angle measurement line 476. For example, in FIG. 4B, the angle measurement lines 476 measuring 10°, 20°, and 300 may be seen at 447a, 447b, and 447c, respectively. In examples, the angle measurement lines 476 may include other indicia, such as notches, markings, and/or any indicator that indicate the different angle measurements. In examples, instead of degree markers, the indicia (e.g., angle measurement lines 476), may further include one or more numbers, letters, and/or symbols. FIG. 4B depicts the angle measurement lines 476 originating at the template center point 464 and emanating away from the edge point 408. The measurement template 460 may be oriented such that the angle measurement lines 476 may emanate in any direction, including toward the edge point 408 (e.g., as depicted in FIGS. 4C and 4D).

As depicted in FIG. 4B, the measurement template 460 may be aligned such that edge 470 of the measurement template 460 is adjacent to the front edge line 404. When the measurement template 460 is aligned properly, edge 470 may line up with the recorded front edge line 404. In FIG. 4B, the edge 470 of the measurement template 460 may be pictured as being nearly adjacent to, but not overlapping, the front edge line 404 for clarity. When in use, the edge 470 and front edge line 404 may overlap and/or be colinear. The center point 472 of the edge 470 (e.g., the point where the width guideline 462b intersects with edge 470 at a right angle), may overlap the edge point 408. In FIG. 4B, the center point 472 may be pictured as being nearly adjacent to, but not overlapping, the edge point 408 for clarity. When in use, the width guideline 462b and the perpendicular marking line 410 may be colinear.

After the measurement template 460 has been aligned properly as discussed above, the motor threshold (MT) position 480 may be recorded on the head covering 430, or, if no head covering 430 is used, the patient's scalp, via inking, marking, and/or similar writing means. The MT position 480 may be marked at the point exposed through the hole at template center point 464.

As shown in FIG. 4C, after identifying and/or recording the MT position 480, the measurement template 460 may be removed from the patient 440's head. The measurement template 460 may then be flipped and/or rotated such that angle measurement lines 476 direct toward the edge point 408 as illustrated in FIG. 4C. The measurement template 460 may remain aligned on the patient 440's head such that the edge 470 of the measurement template 460 and the front edge line 404 overlap and are colinear, and the width guideline 462b and the perpendicular marking line 410 are colinear. In FIG. 4C, the edge 470 of the measurement template 460 may be pictured as being nearly adjacent to, but not overlapping, the front edge line 404 for clarity.

With the measurement template 460 in place, the technician may draw or follow an MT-to-reference line 482, illustrated in FIG. 4C as a dashed line. The MT-to-reference line 482 may extend from the MT position 480 to a reference point (not shown in FIG. 4C), such as the reference point 484 depicted in FIG. 4E. As described in greater detail in FIG. 4E, the reference point (not shown in FIG. 4C), may be on the support structure, position angle measurement instrument, or the patient 440's face, (e.g., the nasion, philtrum, nose, space between the eyes, etc.). FIG. 4C depicts the origin of the MT-to-reference line 482 (e.g., the MT position 480) and the portion of the MT-to-reference line 482 that does not wrap around the patient 440's head and instead terminates at the reference point. The MT-to-reference line 482 may continue to the front of the patient 440's face and/or other anatomical contours as depicted in FIG. 4E.

As seen in FIG. 4D, the technician may shift the measurement template 460 position along the MT-to-reference line 482 in the anterior direction (e.g., toward the front of the patient 440's head). The technician may then move the measurement template 460 along the MT-to-reference line 482 a predetermined distance. After moving this predetermined distance, the template center point 464 (and corresponding hole located at the template center point 464) may overlap the treatment location 490. The predetermined distance may range from 4.0 cm to 6.0 cm (e.g., 5 cm or 5.5 cm).

After the technician shifts the measurement template 460 such that the template center point 464 overlaps the treatment location 490 as discussed above, the treatment location 490 may be recorded on the head covering 430, or, if no head covering 430 is used, the patient's scalp, via inking, marking, and/or similar writing means. The treatment location 490 may be marked at the point exposed through the hole at template center point 464. While the technician moves the measurement template 460 along the MT-to-reference line 482 the predetermined distance to arrive at the treatment location 490, the technician should be careful not to rotate, swivel, or otherwise change the measurement template 460 orientation about the template center point 464.

After the technician shifts the measurement template 460 the predetermined distance, the orientation angle measurement instrument 318 may be placed against the measurement template 460 such that the center of the upright face 422 of the orientation angle measurement instrument 318 contacts the center point 472 of edge 470 of the measurement template 460. Prior to the shifting of the measurement template 460, the orientation angle measurement instrument 318 may be removed and/or disengaged from the patient's 440's head. Additionally or alternatively, the orientation angle measurement instrument 318 may shift along the MT-to-reference line 482 with the measurement template 460 rather than being removed and then re-aligned after the measurement template 460 has shifted. After being placed against the measurement template 460, the orientation angle measurement instrument 318 may measure the orientation angle 494. The orientation angle 494 may be the angle of the position of the treatment coil 350 relative to a reference point (not shown in FIG. 4D), such as the reference point 484 depicted in FIG. 4E. As discussed above, that reference point may lie on the support structure, position angle measurement instrument, or the patient's face, (e.g., the nasion, philtrum, nose, between the eyes, etc.).

In FIG. 4D, the orientation angle 494 is denoted by a dotted arc and measured as the angle between the MT-to-reference line 482 and the treatment location-to-instrument line 474. The treatment location-to-instrument line 474 may be defined by the section of line segment of width guideline 462b between the treatment location 490 (e.g., the center point 464) and the edge 470. It should be noted that the section of the MT-to-reference line 482 used to measure the orientation angle 494 may be measured from the treatment location as depicted in FIG. 4E. While the orientation angle 494 may be measured by using the section of MT-to-reference line 482 that originates from the MT position 480, acquiring the orientation angle 494 measurement by measuring the angle generated from the treatment location 490 will provide greater accuracy in acquiring other measurements (e.g., linear measurement and position angle) as described below. The angle measurement lines 476 may also measure the orientation angle 494 and/or corroborate the measurement taken by the orientation angle measurement instrument 318. The orientation angle 494 may then be taken and/or recorded. In subsequent treatments, the treatment coil 350 may be placed and oriented on the patient's head at the recorded orientation angle 494 without having to remeasure and/or re-establish the MT threshold 480 and/or treatment location 490. In subsequent treatments, the technician may simply place the treatment coil 350 on the patient 440's head and orient the treatment coil 350 at the same orientation angle featured on the orientation angle measurement instrument 318. In examples, the head support guide system may be fitted with a device (not pictured in FIG. 4D) that may allow a user to input coordinates for the MT position 480 (e.g., calculator). In response, the device may display the coordinates of the treatment location 490 based on the inputted coordinates.

FIG. 4E depicts the front view of a human subject 440 prior to and/or undergoing an initial TMS treatment. As shown, the treatment coil 350 may be placed on the patient's head over the MT position 480 (not shown in FIG. 4E) to administer electromagnetic pulses to the treatment location 490 (not shown in FIG. 4E). With the treatment coil 350 in use, the orientation angle measurement instrument 318 may measure the orientation angle (e.g., the angle of the treatment coil's position relative to a reference point 484). The reference point 484 may lie on the support structure, position angle measurement instrument, or the patient's face, (e.g., the nasion, philtrum, nose, etc.). For example, as depicted in FIG. 4E, the reference point 484 may be the nasion of the human subject 440. With the treatment coil 350 in use during the initial treatment, measurements for the linear distance between that of the treatment coil 350 and support structure 302 as well as the position angle of the treatment coil 350 may be taken and/or recorded.

As described above, the position measurement indicator 312 may be received through the first slot 314 the strip 320 of the linear measurement instrument 316. The strip 320 may slide in and out of the first slot 314 as the treatment coil 350 moves about the patient 440's head. When the treatment coil 350 administers treatment, the linear measurement instrument 316 may be colinear with the MT-to-reference line 482, depicted in FIG. 4E as a dashed line. In other words, when observing the patient 440 from a face-to-face perspective, the MT-to-reference line 482 and center line 321 of the strip 320 are colinear.

It should be noted that in FIG. 4E, the center line 321 may not extend all the way to the orientation angle measurement instrument 318. Rather, in FIG. 4E, the center line 321 may terminate at a point before reaching the orientation angle measurement instrument 321, with the remainder of the distance between the last line 332 and the orientation angle measurement instrument 318 depicted as a dashed line for clarity in observing the MT-to-reference line 482. The MT-to-reference line 482 may extend from its origin at the MT position 480 (not shown in FIG. 4E) and continue to the reference point 484 (e.g., the patient 440's nasion).

Just as the process described above may guide a technician to ascertain the orientation angle 494 of the treatment coil 350 about a patient 440's head, so too does the process allow the technician to determine the linear measurement between the treatment coil 350 and support structure 302. The linear measurement instrument 316 may change its position depending on where the treatment coil 350 ends up to properly apply the pulsating magnetic field to the treatment location 494. When the treatment coil 350 is in the proper place to administer treatment, the linear measurement instrument 316 will remain stationary with one end coupled to the orientation angle measurement instrument 316 and its other end fed through the opening of the position measurement indicator 312. With the linear measurement instrument 316 in place, the relevant measurement of linear distance may be determined by observing the lines 332 (or other indicia that may denote measurements along the strip 320) through the first slot 314 and between the distance indicia 317. The linear distance may then be recorded. In subsequent treatments, the technician may simply place the treatment coil 350 on the patient 440's head and orient the treatment coil 350 at the same linear distance established by the linear measurement instrument 316 and viewed through the first slot 314 and distance indicia 317.

Just as the process described above may guide a technician to ascertain the orientation angle 494 of the treatment coil 350 about a patient 440's head, and the linear distance between the treatment coil 350 and support structure 302, so too does the process allow the technician to determine the position angle measurement between the MT-to-reference line 482 and the median plane 446. As described above, the position angle measurement instrument 310 may measure the position angle 444. The position angle 444 is the angle formed by the MT-to-reference line 482 and the median plane 446, depicted in FIG. 4E as a dashed line. The median plane 446 passes through the reference point 484 where it intersects with the MT-to-reference line 482. As seen in FIG. 4E, the median plane 446 is situated perpendicularly in reference to the support structure 302. In other words, the median plane yields a 0° reading on the position angle measurement instrument 310 as seen at 331a.

To measure the position angle 444, the technician may observe where the center line 321 of the strip 320 passes over the position angle measurement instrument 310. The center lines will pass over the position angle lines 330. In examples, the strip 320 is transparent, such that the technician may see the position angle lines 330 angle indicia 319 printed on the position angle measurement instrument 310 through the strip 320. The measurement identified by the angle indicia 319 relative to where the center line 321 of the strip 320 passes over position angle lines 330 may also denote the position angle 444. For example, if the linear measurement instrument 316 is oriented perpendicular to the support structure 302, then the strip 320 and its center line 321 will pass over position angle lines 330 at 0° as seen at 331a. The position angle 444 may then be recorded. In subsequent treatments, the technician may simply place the treatment coil 350 on the patient 440's head and orient the treatment coil 350 at the same position angle 444 established by the position angle measurement instrument 316 and observed by angle indicia 319.

Additionally or alternatively, the reference point 484 may be located on the support structure 302, position angle measurement instrument 310, or the patient's face such that a plane passing through the reference point 484 and situated perpendicularly in reference to the support structure does not precisely divides the human body into left and right sides of equal proportion. In this example, the plane is a sagittal or longitudinal plane and the position angle may still be calculated by the same methods described herein.

In FIG. 4E, the position angle 444 is denoted by a dashed arc. The position angle measurement instrument 310 may measure the position angle 444 from the median plane 446 and extending to the MT-to-reference line 482. The position angle 444 measurement may be indicated and/or read by the lines 332, notches, markings, and/or any indicator that denotes different angle measurements on the position angle measurement instrument 530. The position angle measuring instrument 330 may be made of a thin, flexible, and/or clear material (e.g., plastic) for easier reading of the lines 332 (or, e.g., markings, notches, and/or any indicator that denotes measurements along the strip). The position angle 444 may then be recorded.

FIG. 5 is a flowchart 500 depicting the operation of the head support guide and method of obtaining critical measurements that obviate the need for repeated use of the head support guide. At 502, the process may begin when the technician initially determines the patient's MT position. The technician may then record the MT position, either in a record book, record keeping software, and/or on a head covering worn by the patient during the initial treatment.

At 504, the technician may then determine the treatment location. As described herein, the technician may begin the process of obtaining the treatment location by placing the treatment coil on the patient's head over the predetermined MT position. The technician may mark the front edge of the treatment coil on the head covering. The technician may mark on the line denoting the front edge of the treatment coil, of the center point of the treatment coil (e.g., where the front edge line and center point intersect). With the markings made on the head covering, the treatment coil may be removed. The treatment coil may be replaced by the measurement template. The measurement template may be aligned along the recorded front edge line. The technician may mark MT position on the head covering through the hole at the center of the measurement template. Then, a reference point may be established. The MT position-to-reference line may then be constructed connecting the MT position and reference. With the MT position-to-reference line drawn, the measurement template may be shifted along the MT-to-reference line a predetermined difference (e.g., 4.0 cm to 6.0 cm). When the measurement template is shifted this predetermined difference, the resulting location at the center of the measurement template may mark the treatment location. The technician may mark and/or record the treatment location through the center hole of the now-shifted measurement template in a manner described herein.

At 506, the technician may next determine the orientation angle. The technician may construct a line connecting the treatment location and where the center point of the measurement template meets the orientation angle measurement instrument (e.g., the treatment location-to-instrument line). The technician may mark the treatment location-to-instrument line on the head covering.

With the proper lines measured and/or established, the technician may then use the orientation angle measurement instrument to measure the angle between the treatment location-to-reference line and the MT-to-reference line. The technician may measure from where the MT-to-reference line crosses the treatment location rather than the MT-to-reference line's origin at the MT position. The technician may then mark and/or record the orientation angle in a manner described herein.

At 508, the technician may next determine the linear distance. The technician may affix one end of the strip of the linear measurement instrument to the orientation angle measurement instrument. The other end of the strip (e.g., non-affixed end) may then be fed through the position measurement indicator. The technician may obtain the linear measurement by observing the indicia printed on the strip that shows through the first slot of the position measurement indicator. The technician may then mark and/or record the linear measurement in a manner described herein.

At 510, the technician may next determine the position angle. The technician may press the strip against the position angle measurement indicator (if necessary) such that the indicia printed on the position measurement angle instrument are visible through the strip. The technician may next observe the center line printed on the strip passes over the indicia printed on the position angle measurement instrument (e.g., the MT-to-reference line). The technician may identify the median plane that passes through the patient (e.g., the imaginary plane that bisects the patient into a left half and a right half). The technician may use the position angle measurement instrument to measure the angle between the MT-to-reference line and the median plane. The technician may then mark and/or record the position angle in a manner described herein.

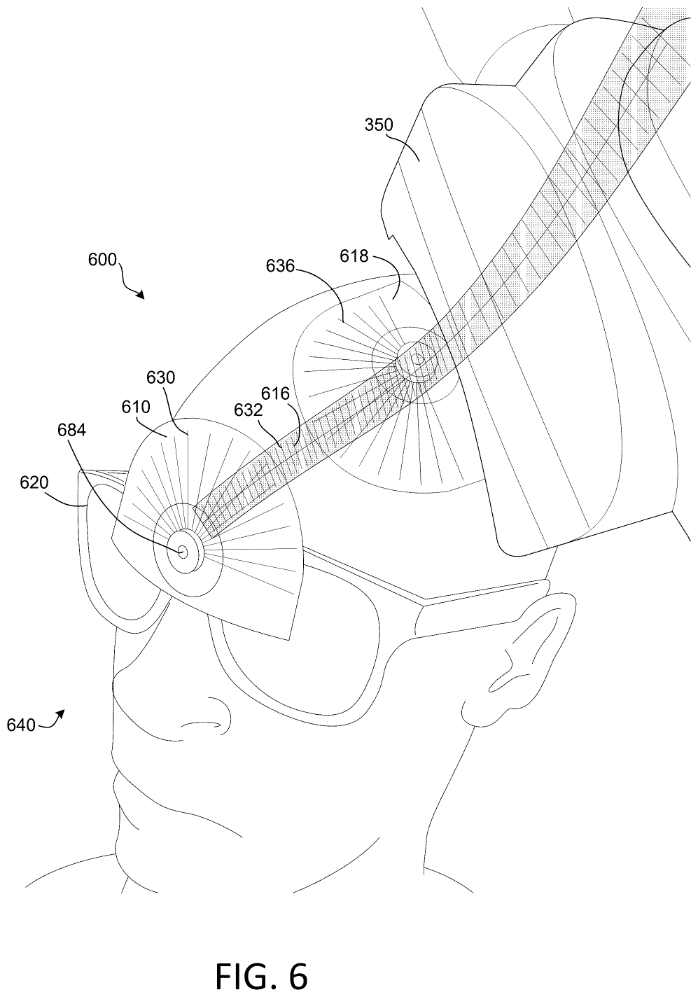

FIG. 6 depicts an alternative head support guide system 600 wherein the patient 640 uses removable eyeglasses 620 (e.g., the patient 640's own prescription eyeglasses, reading glasses, personal sunglasses, etc.) in lieu of a support structure. The head support guide system 600 may include a plurality of measurement instruments, such as a position angle measurement instrument 610, a linear measurement instrument 616, and an orientation angle measurement instrument 618. The position angle measurement instrument 610 may be configured to indicate a position angle for placement of the treatment coil 350. The linear measurement instrument 316 may be configured to indicate a distance from the support structure 302 for placement of the treatment coil 350. An alternative orientation angle measurement instrument 618 may be configured to indicate an orientation angle for rotation of the treatment coil 350. Accordingly, the head support guide 600 may be configured to measure the distance, position angle, and/or the orientation angle of the treatment coil 350 when the treatment coil 350 is located at the treatment location for the patient (e.g., and/or located at the MT position of the patient), for example, using a method similar to that described with reference to FIGS. 4A-E and/or FIG. 5.

In this example, the position angle measurement instrument 610 may be placed at the center of the bridge of the eyeglasses 620. The position angle measurement instrument 610 may be fixed to the eyeglasses 620 with tape, adhesive, and/or hardware that may temporarily connect the position angle measurement instrument 610 to the eyeglasses 620. The reference point 684 (e.g., the point of intersection where the MT-to-reference line and median plane intersect) may be located at the nasion of the human subject 640. As depicted in FIG. 6, the reference point 684 and the center of the bridge of the eyeglasses 620 that rests on the nasion of the patient 640 may be the same point. The measurement of the linear distance, the position angle, and orientation angle may be taken in the same manner as described above. The MT position (not pictured in FIG. 6) may lie under the treatment coil 350. From the MT position, the MT-to-reference line may be ascertained in the same manner as described above. With the construction of the MT-to-reference line, the technician may measure and/or record the linear distance, position angle, and/or the reference angle.

The position angle measurement instrument 610 and the orientation angle measurement instrument 618 are depicted in FIG. 6 as protractors featuring bold indicia, e.g. position angle lines 630 and orientation angle lines 636, respectively. The position angle lines 630 and orientation angle lines 636 may be used for reading the position angle and orientation angle, respectively. These large protractors may be made of thin, flexible, and/or transparent material (e.g., plastic) for easier reading of the position and/or orientation angles. The position angle lines 630 and orientation angle lines 636 may be featured in dark print to contrast from the thin, flexible, and/or transparent protractors for easier reading of the measured angles.

The linear measurement instrument 616 may be made of thin, flexible, and/or transparent material (e.g., plastic) featuring bold indicia (e.g., lines 632) for easier reading and/or measurement of the linear distance. The lines 632 may be featured in dark print to contrast from the thin, flexible, and/or transparent linear measurement instrument 616 for easier reading of the measured linear distance.

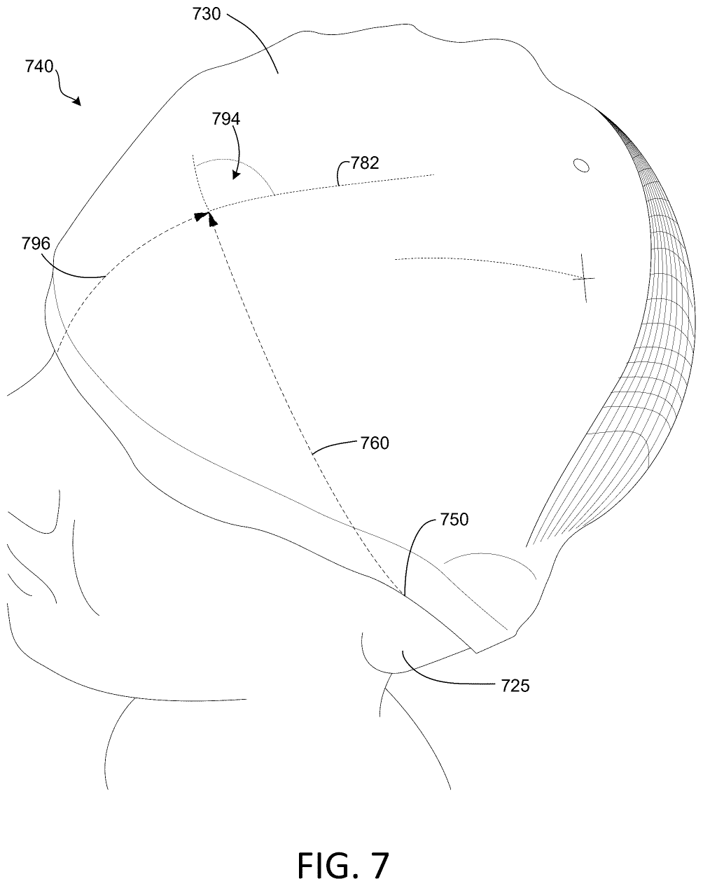

FIG. 7 depicts an alternative method for establishing measurements for reproducing placement of the treatment coil (not shown in FIG. 7). Measurements may be recorded on the head covering 730 (e.g., a skull cap) worn by the patient 740. The measurements for recording include the orientation angle 794 and a first linear distance 796. The orientation angle 794 and the first linear distance 796 measurements may be obtained in the same manner as in the embodiments previously discussed. However, in lieu of a position angle, a second reference point 750 may be used to establish a second linear distance 760 as a measurement for treatment coil placement. The second reference point 750 may originate from the side of the head of the patient 740. The second linear distance 760 may be measured from the second reference point 750, depicted in FIG. 7 as lying on the left ear 725 of the patient 740. The second linear distance 760 may extend from the second reference point 750 to a point where it meets the MT-to-reference line 782. The second linear distance 760 may then be recorded in the same manner as described above.

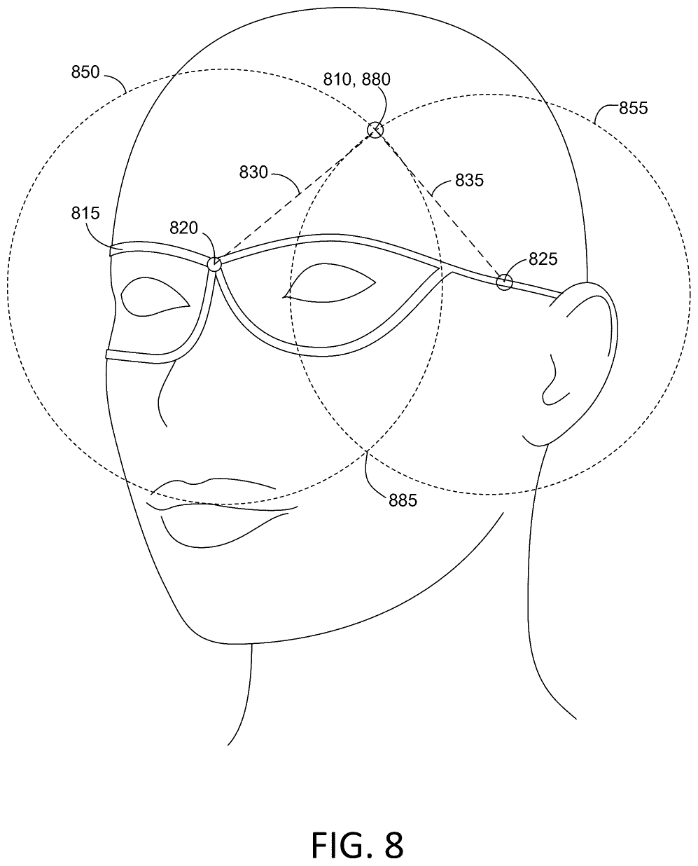

FIG. 8 depicts another alternative method for establishing measurements for reproducing placement of the treatment coil (not shown in FIG. 8). On initial use, after the physician and/or technician has ascertained the treatment location, the physician and/or technician may make two measurements. The first measurement may be taken from the treatment location 810 to a first reference point 820 located on the support structure 815 and defining a first treatment-location-to-reference-point line 830. Though depicted in FIG. 8 as on the support structure resting on patient's nasion, the first reference point 820 may be on the support structure or elsewhere on the patient 440's face, (e.g., the philtrum, nose, space between the eyes, etc.). Then, a second measurement may be taken from the treatment location 980 to a second reference point 825 located on an arm bar 806 of the head support guide and defining a second treatment-location-to-reference-point line 835. The physician and/or technician may then record the first and second treatment-location-to-reference-point lines 830, 835. Further, the physician and/or technician may then record the location of the first and second reference points 820, 825 (e.g., by marking the head support guide).

To recreate the treatment location on subsequent use, a first circular reference area 850 may be ascertained by generating a circle at the first reference point 820, wherein the first reference area 850 has a radius equal to the first treatment-location-to-reference-point line 830. The process may then be repeated at the second reference point 835. A second circular reference area 855 may be ascertained by generating a circle at the second reference point 825, wherein the second reference area 855 has a radius equal to the second treatment-location-to-reference-point line 835.

With first and second circular reference areas 850, 855 generated, the two circular reference areas 850, 855 will intersect at first and second reference intersection points 880, 885. The second reference intersection point 885 is not considered the treatment location as it exists beneath the brain on the lower side of the patient's face. Therefore, the first intersection point 880 may be the treatment location.

It should be appreciated that the example head support guide system use in conjunction with TMS devices described herein are not limited to their illustrated configurations. For example, one or more components from a first one of the example head support guide systems may be implemented in a second one of the example head support guide systems. One of ordinary skill in the art will appreciate that these and other different configurations of the example head support guide system may be implemented without departing from the scope and spirit of the instant disclosure.

What is claimed is:

1. A coil positioning device for positioning a transcranial magnetic stimulation (TMS) coil of a TMS system relative to a patient's head, the coil positioning device comprising:
a support structure configured to be supported by the patient's head;
a first instrument configured to indicate a position angle for placement of the TMS coil;
a second instrument configured to indicate a distance from the support structure for placement of the TMS coil; and
a third instrument configured to indicate an orientation angle for rotation of the TMS coil.

2. The coil positioning device of claim 1, wherein the coil positioning device is configured to identify a location of the TMS coil for TMS treatment based on the distance and the position angle relative to a reference point defined by the support structure.

3. The coil positioning device of claim 2, wherein the coil positioning device is configured to identify an orientation of the TMS coil for TMS treatment based on the orientation angle.

4. The coil positioning device of claim 2, wherein the reference point is a motor threshold (MT) location.

5. The coil positioning device of claim 1, wherein the coil positioning device is configured to identify a motor threshold location associated with TMS treatment based on the distance and the position angle relative to a reference point defined by the support structure.

6. The coil positioning device of claim 1, wherein the support structure comprises:
a first elongated arm configured to be supported by a first ear of the patient;
a second elongated arm configured to be supported by a second ear of the patient; and
a crossbar residing between the first elongated arm and the second elongated arm.

7. The coil positioning device of claim 6, wherein the support structure, the crossbar, or the first instrument defines a reference point for measuring the position angle, the distance, and the orientation angle.

8. The coil positioning device of claim 6, wherein the crossbar is connected to the first elongated arm by a first hinge and the second elongated arm by a second hinge.

9. The coil positioning device of claim 6, wherein the first instrument is coupled to the crossbar.

10. The coil positioning device of claim 9, wherein the first instrument is coupled to the crossbar at the reference point.

11. The coil positioning device of claim 1, wherein the third instrument further comprises a removable template for measuring the orientation angle, wherein the template facilitates repositioning of the TMS coil when the template is attached.

12. The coil positioning device of claim 1, wherein the third instrument is coupled to a distal end of the second instrument, wherein the third instrument comprises a reference point for measuring the orientation angle.

13. The coil positioning device of claim 1, wherein the second instrument is a tape measure.

14. The coil positioning device of claim 1, wherein the distance is an arc length.

15. A device for positioning a transcranial magnetic stimulation (TMS) coil of a TMS system relative to a patient's head, the device comprising:
a support structure configured to be support by the patient's head, wherein the support structure comprises:
a first elongated arm;
a second elongated arm; and
a crossbar residing between the first elongated arm and the second elongated arm, wherein the crossbar comprises a first instrument for measuring a position angle for placement of the TMS coil to conduct a TMS treatment session, and wherein the crossbar defines a reference point for measuring the position angle; and
a second instrument coupled to the support structure, wherein the second instrument is for measuring a distance from the support structure to the TMS coil to conduct the TMS treatment session, wherein the second instrument comprises a third instrument at a distal end of the second instrument for measuring an orientation angle of the TMS coil to conduct the TMS treatment session.

16. The device of claim 15, wherein the position angle represents an angular offset between a line located within a median plane of the patient that intersects with the reference point and a line that intersects the reference point and a motor threshold location of the patient.

17. The device of claim 15, wherein the orientation angle represents an angular offset between a line that intersects the motor threshold location of the patient and the TMS and a line that intersects the reference point and the motor threshold location of the patient.

18. The device of claim 15, wherein the third instrument defines a flat edge; and
wherein the orientation angle represents an angular offset between a line that intersects the flat side of the third instrument and the motor threshold location of the patient and a line that intersects the motor threshold location of the patient and the TMS coil (e.g., an edge of the TMS coil) and a line that intersects the reference point and the motor threshold location of the patient.

19. The device of claim 18, wherein the flat edge is configured to rest next to an edge of the TMS coil.

20. The device of claim 15, wherein the support structure is configured to align the reference point with the patient's nasion.

21.-62. (canceled)
